SEQUENCE LISTING (116 sequences)

SEQ ID NO: 1 - DNA, 285 bp, Artificial Sequence
  fusion gene
cctctctcccctccgaccaatctccatctcgaagcaaaccctgacaccggggtcctcacggtttcatgggaaagaagtaccacgccggatataacgggttaccgcataacgaccacgccaacgaacgggcaacagggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgtt

SEQ ID NO: 2 - protein, 95 aa, Artificial Sequence
  fusion protein
PLSPPTNLHLEANPDTGVLTVSWERSTTPDITGYRITTTPTNGQQGNSLEEVVHADQSSCTFDNLSPGLEYNVSVYTVKDDKESVPISDTIIPAV

SEQ ID NO: 3 - DNA, 282 bp, Artificial Sequence
  fusion gene
gtaagcgacgtcccccgagacctggaagtcgtggccgccacacccacttccctccttatctcttgggacgcacccgctgtcaccgttcggtattacaggatcacatatggagagacgggcggaaatagccccgtccaagagtttaccgtccctgggagtaaaagtacagccactataagtggcctcaaacctggtgttgattatacgatcaccgtctatgctgtgacggggagaggagatagtccagcgtcttcaaaacccattagcatcaattatcggacc

SEQ ID NO: 4 - protein, 94 aa, Artificial Sequence
  fusion protein
VSDVPRDLEVVAATPTSLLISWDAPAVTVRYYRITYGETGGNSPVQEFTVPGSKSTATISGLKPGVDYTITVYAVTGRGDSPASSKPISINYRT

SEQ ID NO: 5 - DNA, 522 bp, Artificial Sequence
  fusion gene
acacctctgggccccgcctcctccctgcctcagagctttctgctcaaatgtctggagcaggtgcggaagatccagggcgacggcgccgctctgcaagagaaactgtgcgccacatataagctgtgtcaccccgaggaactggtcctcttgggccacagcctgggcatcccctgggcccctctcagctcctgcccctcccaagctctccaactggctggatgtctgtcccaactgcactccggcctcttcctgtaccagggactcctccaggctctcgaagggatcagccccgaactgggccccacactggacaccttgcaactcgatgtggccgatttcgccacaaccatctggcagcagatggaagaactcggaatggctcctgctctccagcccacacagggagctatgcctgctttcgcctctgctttccagcggagagctggtggtgtgctcgtcgcatcccacctccagagcttcttggaggtgtcctatcgggtgctccggcatctggcccaaccc

SEQ ID NO: 6 - protein, 174 aa, Artificial Sequence
  fusion protein
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 7 - DNA, 573 bp, Artificial Sequence
  fusion gene
ttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttc

SEQ ID NO: 8 - protein, 191 aa, Artificial Sequence [sequence omitted]

SEQ ID NO: 9 - DNA, 942 bp, Artificial Sequence
  fusion gene
gtaagcgacgtcccccgagacctggaagtcgtggccgccacacccacttccctccttatctcttgggacgcacccgctgtcaccgttcggtattacaggatcacatatggagagacgggcggaaatagccccgtccaagagtttaccgtccctgggagtaaaagtacagccactataagtggcctcaaacctggtgttgattatacgatcaccgtctatgctgtgacggggagaggaggaggaagtggtggtggggggagcggtggtggtggaagcggaggcggaggctcaacacctctgggccccgcctcctccctgcctcagagctttctgctcaaatgtctggagcaggtgcggaagatccagggcgacggcgccgctctgcaagagaaactgtgcgccacatataagctgtgtcaccccgaggaactggtcctcttgggccacagcctgggcatcccctgggcccctctcagctcctgcccctcccaagctctccaactggctggatgtctgtcccaactgcactccggcctcttcctgtaccagggactcctccaggctctcgaagggatcagccccgaactgggccccacactggacaccttgcaactcgatgtggccgatttcgccacaaccatctggcagcagatggaagaactcggaatggctcctgctctccagcccacacagggagctatgcctgctttcgcctctgctttccagcggagagctggtggtgtgctcgtcgcatcccacctccagagcttcttggaggtgtcctatcgggtgctccggcatctggcccaacccggtggaggcgggagtggcggtgggtctggtggcggtggtagtgggggttcaggtgatagtccagcgtcttcaaaacccattagcatcaattatcggaccggcggaggccaccaccatcatcaccatcac

SEQ ID NO: 10 - protein, 314 aa, Artificial Sequence
  fusion protein
VSDVPRDLEVVAATPTSLLISWDAPAVTVRYYRITYGETGGNSPVQEFTVPGSKSTATISGLKPGVDYTITVYAVTGRGGGSGGGGSGGGGSGGGGSTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGSGGGGSGGSGDSPASSKPISINYRTGGGHHHHHHH

SEQ ID NO: 11 - DNA, 948 bp, Artificial Sequence
  fusion gene
cctctctcccctccgaccaatctccatctcgaagcaaaccctgacaccggggtcctcacggtttcatgggaaagaagtaccacgccggatataacgggttaccgcataacgaccacgccaacgaacgggggaggaagtggtggtggggggagcggtggtggtggaagcggaggcggaggctcaacacctctgggccccgcctcctccctgcctcagagctttctgctcaaatgtctggagcaggtgcggaagatccagggcgacggcgccgctctgcaagagaaactgtgcgccacatataagctgtgtcaccccgaggaactggtcctcttgggccacagcctgggcatcccctgggcccctctcagctcctgcccctcccaagctctccaactggctggatgtctgtcccaactgcactccggcctcttcctgtaccagggactcctccaggctctcgaagggatcagccccgaactgggccccacactggacaccttgcaactcgatgtggccgatttcgccacaaccatctggcagcagatggaagaactcggaatggctcctgctctccagcccacacagggagctatgcctgctttcgcctctgctttccagcggagagctggtggtgtgctcgtcgcatcccacctccagagcttcttggaggtgtcctatcgggtgctccggcatctggcccaacccggtggaggcgggagtggcggtgggtctggtggcggtggtagtgggggttcaggtggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggcggaggccaccaccatcatcaccatcac

SEQ ID NO: 12 - protein, 316 aa, Artificial Sequence
  fusion protein
PLSPPTNLHLEANPDTGVLTVSWERSTTPDITGYRITTTPTNGGGSGGGGSGGGGSGGGGSTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGSGGGGSGGSGGNSLEEVVHADQSSCTFDNLSPGLEYNVSVYTVKDDKESVPISDTIIPAVGGGGGGHHHHHHH

SEQ ID NO: 13 - DNA, 1605 bp, Artificial Sequence
  fusion gene
cctctctcccctccgaccaatctccatctcgaagcaaaccctgacaccggggtcctcacggtttcatgggaaagaagtaccacgccggatataacgggttaccgcataacgaccacgccaacgaacgggggaggaagtggtggtggggggagcggtggtggtggaagcggaggcggaggctcaacacctctgggccccgcctcctccctgcctcagagctttctgctcaaatgtctggagcaggtgcggaagatccagggcgacggcgccgctctgcaagagaaactgtgcgccacatataagctgtgtcaccccgaggaactggtcctcttgggccacagcctgggcatcccctgggcccctctcagctcctgcccctcccaagctctccaactggctggatgtctgtcccaactgcactccggcctcttcctgtaccagggactcctccaggctctcgaagggatcagccccgaactgggccccacactggacaccttgcaactcgatgtggccgatttcgccacaaccatctggcagcagatggaagaactcggaatggctcctgctctccagcccacacagggagctatgcctgctttcgcctctgctttccagcggagagctggtggtgtgctcgtcgcatcccacctccagagcttcttggaggtgtcctatcgggtgctccggcatctggcccaacccggtggaggcgggagtggcggtgggtctggtggcggtggtagtgggggttcaggtggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggaggcggagacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - protein, 535 aa, Artificial Sequence
  fusion protein
PLSPPTNLHLEANPDTGVLTVSWERSTTPDITGYRITTTPTNGGGSGGGGSGGGGSGGGGSTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGSGGGGSGGSGGNSLEEVVHADQSSCTFDNLSPGLEYNVSVYTVKDDKESVPISDTIIPAVGGGGGGDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 999 bp, Artificial Sequence
  fusion gene
cctctctcccctccgaccaatctccatctcgaagcaaaccctgacaccggggtcctcacggtttcatgggaaagaagtaccacgccggatataacgggttaccgcataacgaccacgccaacgaacgggggaggaagtggtggtggggggagcggtggtggtggaagcggaggcggaggctcattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggtggaggcgggagtggcggtgggtctggtggcggtggtagtgggggttcaggtggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggcggaggccaccaccatcatcaccatcac

SEQ ID NO: 16 - protein, 333 aa, Artificial Sequence
  fusion protein
PLSPPTNLHLEANPDTGVLTVSWERSTTPDITGYRITTTPTNGGGSGGGGSGGGGSGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGSGGGGSGGSGGNSLEEVVHADQSSCTFDNLSPGLEYNVSVYTVKDDKESVPISDTIIPAVGGGGGGHHHHHHH

SEQ ID NO: 17 - DNA, 1656 bp, Artificial Sequence
  fusion gene
cctctctcccctccgaccaatctccatctcgaagcaaaccctgacaccggggtcctcacggtttcatgggaaagaagtaccacgccggatataacgggttaccgcataacgaccacgccaacgaacgggggaggaagtggtggtggggggagcggtggtggtggaagcggaggcggaggctcattcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggtggaggcgggagtggcggtgggtctggtggcggtggtagtgggggttcaggtggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggaggcggagacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 18 - protein, 552 aa, Artificial Sequence
  fusion protein
PLSPPTNLHLEANPDTGVLTVSWERSTTPDITGYRITTTPTNGGGSGGGGSGGGGSGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSGGGSGGGGSGGSGGNSLEEVVHADQSSCTFDNLSPGLEYNVSVYTVKDDKESVPISDTIIPAVGGGGGGDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 978 bp, Artificial Sequence
  fusion gene
cctctctcccctccgaccaatctccatctcgaagcaaaccctgacaccggggtcctcacggtttcatgggaaagaagtaccacgccggatataacgggttaccgcataacgaccacgccaacgaacgggggcggaagcggagcaaagctcgccgcactgaaagccaagctggccgctctgaagggaggtggcgggagcacacctctgggccccgcctcctccctgcctcagagctttctgctcaaatgtctggagcaggtgcggaagatccagggcgacggcgccgctctgcaagagaaactgtgcgccacatataagctgtgtcaccccgaggaactggtcctcttgggccacagcctgggcatcccctgggcccctctcagctcctgcccctcccaagctctccaactggctggatgtctgtcccaactgcactccggcctcttcctgtaccagggactcctccaggctctcgaagggatcagccccgaactgggccccacactggacaccttgcaactcgatgtggccgatttcgccacaaccatctggcagcagatggaagaactcggaatggctcctgctctccagcccacacagggagctatgcctgctttcgcctctgctttccagcggagagctggtggtgtgctcgtcgcatcccacctccagagcttcttggaggtgtcctatcgggtgctccggcatctggcccaacccggcggaggtgggagtgaactggccgcactggaagctgagctggctgccctcgaagctggaggctctggaggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggaggcggacaccaccatcatcaccatcac

SEQ ID NO: 20 - protein, 326 aa, Artificial Sequence
  fusion protein
PLSPPTNLHLEANPDTGVLTVSWERSTTPDITGYRITTTPTNGGGSGAKLAALKAKLAALKGGGGSTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSELAALEAELAALEAGGSGGNSLEEVVHADQSSCTFDNLSPGLEYNVSVYTVKDDKESVPISDTIIPAVGGGGGGHHHHHHH

SEQ ID NO: 21 - DNA, 1029 bp, Artificial Sequence
  fusion gene
cctctctcccctccgaccaatctccatctcgaagcaaaccctgacaccggggtcctcacggtttcatgggaaagaagtaccacgccggatataacgggttaccgcataacgaccacgccaacgaacgggggcggaagcggagcaaagctcgccgcactgaaagccaagctggccgctctgaagggaggtggcgggagcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggcggaggtgggagtgaactggccgcactggaagctgagctggctgccctcgaagctggaggctctggaggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggcggaggccaccaccatcatcaccatcac

SEQ ID NO: 22 - protein, 343 aa, Artificial Sequence
  fusion protein
PLSPPTNLHLEANPDTGVLTVSWERSTTPDITGYRITTTPTNGGGSGAKLAALKAKLAALKGGGGSFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGGGSELAALEAELAALEAGGSGGNSLEEVVHADQSSCTFDNLSPGLEYNVSVYTVKDDKESVPISDTIIPAVGGGGGGHHHHHHH

SEQ ID NO: 23 - DNA, 942 bp, Artificial Sequence
  fusion gene
gtaagcgacgtcccccgagacctggaagtcgtggccgccacacccacttccctccttatctcttgggacgcacccgctgtcaccgttcggtattacaggatcacatatggagagacgggcggaggaagtggtggtggggggagcggtggtggtggaagcggaggcggaggctcaacacctctgggccccgcctcctccctgcctcagagctttctgctcaaatgtctggagcaggtgcggaagatccagggcgacggcgccgctctgcaagagaaactgtgcgccacatataagctgtgtcaccccgaggaactggtcctcttgggccacagcctgggcatcccctgggcccctctcagctcctgcccctcccaagctctccaactggctggatgtctgtcccaactgcactccggcctcttcctgtaccagggactcctccaggctctcgaagggatcagccccgaactgggccccacactggacaccttgcaactcgatgtggccgatttcgccacaaccatctggcagcagatggaagaactcggaatggctcctgctctccagcccacacagggagctatgcctgctttcgcctctgctttccagcggagagctggtggtgtgctcgtcgcatcccacctccagagcttcttggaggtgtcctatcgggtgctccggcatctggcccaacccggtggaggcgggagtggcggtgggtctggtggcggtggtagtgggggttcaggtggaaatagccccgtccaagagtttaccgtccctgggagtaaaagtacagccactataagtggcctcaaacctggtgttgattatacgatcaccgtctatgctgtgacggggagaggagatagtccagcgtcttcaaaacccattagcatcaattatcggaccggcggaggccaccaccatcatcaccatcac

SEQ ID NO: 24 - protein, 314 aa, Artificial Sequence
  fusion protein
VSDVPRDLEVVAATPTSLLISWDAPAVTVRYYRITYGETGGGSGGGGSGGGGSGGGGSTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGSGGGGSGGSGGNSPVQEFTVPGSKSTATISGLKPGVDYTITVYAVTGRGDSPASSKPISINYRTGGGHHHHHHH

SEQ ID NO: 25 - DNA, 972 bp, Artificial Sequence
  fusion gene
gtaagcgacgtcccccgagacctggaagtcgtggccgccacacccacttccctccttatctcttgggacgcacccgctgtcaccgttcggtattacaggatcacatatggagagacgggcggcggaagcggagcaaagctcgccgcactgaaagccaagctggccgctctgaagggaggtggcgggagcacacctctgggccccgcctcctccctgcctcagagctttctgctcaaatgtctggagcaggtgcggaagatccagggcgacggcgccgctctgcaagagaaactgtgcgccacatataagctgtgtcaccccgaggaactggtcctcttgggccacagcctgggcatcccctgggcccctctcagctcctgcccctcccaagctctccaactggctggatgtctgtcccaactgcactccggcctcttcctgtaccagggactcctccaggctctcgaagggatcagccccgaactgggccccacactggacaccttgcaactcgatgtggccgatttcgccacaaccatctggcagcagatggaagaactcggaatggctcctgctctccagcccacacagggagctatgcctgctttcgcctctgctttccagcggagagctggtggtgtgctcgtcgcatcccacctccagagcttcttggaggtgtcctatcgggtgctccggcatctggcccaacccggcggaggtgggagtgaactggccgcactggaagctgagctggctgccctcgaagctggaggctctggaggaaatagccccgtccaagagtttaccgtccctgggagtaaaagtacagccactataagtggcctcaaacctggtgttgattatacgatcaccgtctatgctgtgacggggagaggagatagtccagcgtcttcaaaacccattagcatcaattatcggaccggcggaggccaccaccatcatcaccatcac

SEQ ID NO: 26 - protein, 324 aa, Artificial Sequence
  fusion protein
VSDVPRDLEVVAATPTSLLISWDAPAVTVRYYRITYGETGGGSGAKLAALKAKLAALKGGGGSTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSELAALEAELAALEAGGSGGNSPVQEFTVPGSKSTATISGLKPGVDYTITVYAVTGRGDSPASSKPISINYRTGGGHHHHHHH

SEQ ID NO: 27 - DNA, 954 bp, Artificial Sequence
  fusion gene
cctctctcccctccgaccaatctccatctcgaagcaaaccctgacaccggggtcctcacggtttcatgggaaagaagtaccacgccggatataacgggttaccgcataacgaccacgccaacgaacgggcaacagggcaactcacttgaggaggtcgttcatgccgatcaatcttcctgtacgtttgataatttgagtccgggacttgagtacaatgtcagtgtatataccgttaaaggaggaagtggtggtggggggagcggtggtggtggaagcggaggcggaggctcaacacctctgggccccgcctcctccctgcctcagagctttctgctcaaatgtctggagcaggtgcggaagatccagggcgacggcgccgctctgcaagagaaactgtgcgccacatataagctgtgtcaccccgaggaactggtcctcttgggccacagcctgggcatcccctgggcccctctcagctcctgcccctcccaagctctccaactggctggatgtctgtcccaactgcactccggcctcttcctgtaccagggactcctccaggctctcgaagggatcagccccgaactgggccccacactggacaccttgcaactcgatgtggccgatttcgccacaaccatctggcagcagatggaagaactcggaatggctcctgctctccagcccacacagggagctatgcctgctttcgcctctgctttccagcggagagctggtggtgtgctcgtcgcatcccacctccagagcttcttggaggtgtcctatcgggtgctccggcatctggcccaacccggtggaggcgggagtggcggtgggtctggtggcggtggtagtgggggttcaggtgacgataaggagtcagtaccaatcagcgataccattattccggccgttggaggcgggggaggcggacaccaccatcatcaccatcac

SEQ ID NO: 28 - protein, 318 aa, Artificial Sequence
  fusion protein
PLSPPTNLHLEANPDTGVLTVSWERSTTPDITGYRITTTPTNGQQGNSLEEVVHADQSSCTFDNLSPGLEYNVSVYTVKGGSGGGGSGGGGSGGGGSTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGSGGGGSGGSGDDKESVPISDTIIPAVGGGGGGHHHHHHH

SEQ ID NO: 29 - DNA, 972 bp, Artificial Sequence
  fusion gene
gtaagcgacgtcccccgagacctggaagtcgtggccgccacacccacttccctccttatctcttgggacgcacccgctgtcaccgttcggtattacaggatcacatatggagagacgggcggaaatagccccgtccaagagtttaccgtccctgggagtaaaagtacagccactataagtggcctcaaacctggtgttgattatacgatcaccgtctatgctgtgacggggagaggaggcggaagcggagcaaagctcgccgcactgaaagccaagctggccgctctgaagggaggtggcgggagcacacctctgggccccgcctcctccctgcctcagagctttctgctcaaatgtctggagcaggtgcggaagatccagggcgacggcgccgctctgcaagagaaactgtgcgccacatataagctgtgtcaccccgaggaactggtcctcttgggccacagcctgggcatcccctgggcccctctcagctcctgcccctcccaagctctccaactggctggatgtctgtcccaactgcactccggcctcttcctgtaccagggactcctccaggctctcgaagggatcagccccgaactgggccccacactggacaccttgcaactcgatgtggccgatttcgccacaaccatctggcagcagatggaagaactcggaatggctcctgctctccagcccacacagggagctatgcctgctttcgcctctgctttccagcggagagctggtggtgtgctcgtcgcatcccacctccagagcttcttggaggtgtcctatcgggtgctccggcatctggcccaacccggcggaggtgggagtgaactggccgcactggaagctgagctggctgccctcgaagctggaggctctggagatagtccagcgtcttcaaaacccattagcatcaattatcggaccggcggaggccaccaccatcatcaccatcac

SEQ ID NO: 30 - protein, 324 aa, Artificial Sequence
  fusion protein
VSDVPRDLEVVAATPTSLLISWDAPAVTVRYYRITYGETGGNSPVQEFTVPGSKSTATISGLKPGVDYTITVYAVTGRGGGSGAKLAALKAKLAALKGGGGSTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSELAALEAELAALEAGGSGDSPASSKPISINYRTGGGHHHHHHH

SEQ ID NO: 31 - DNA, 687 bp, Artificial Sequence
  fusion gene
gaggtccagctgcagcagagtggtcctgaactggttaagcctggggcatcaatgaaaatctcctgtaaagcaagtggttattccttcaccggctatacaatgaactgggtgaagcagtctcacggaaaaaacctggaatggatggggctgattaatccgtataagggtgttagcacctacaaccagaaattcaaagataaggcaacactgactgtcgacaaaagctcctctaccgcttatatggaactgctgagcctgacatccgaggattctgccgtttattactgcgcgcgcagcggttattacggggattccgactggtactttgacgtgtggggccagggtaccacactgaccgttttcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcac

SEQ ID NO: 32 - protein, 229 aa, Artificial Sequence
  fusion protein
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 33 - DNA, 642 bp, Artificial Sequence
  fusion gene
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 34 - protein, 214 aa, Artificial Sequence
  fusion protein
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - DNA, 681 bp, Artificial Sequence
  fusion gene
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgcggtggggaggtgacggcttctatgccatggactactggggccaaggaaccctggtcaccgtctcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcac

SEQ ID NO: 36 - protein, 227 aa, Artificial Sequence
  fusion protein
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 37 - DNA, 642 bp, Artificial Sequence
  fusion gene
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 38 - protein, 214 aa, Artificial Sequence
  fusion protein
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 1821 bp, Artificial Sequence
  fusion gene
gacattcaaatgacgcagtcaccctcttccctgtccgccagcgtgggggatcgcgtcacaatcacatgtcgcgcctctcaggatgtgaacaccgcggtggcttggtatcaacagaagccaggcaaagcacctaagctcctgatctactctgccagctttttgtacagcggcgtgccaagtaggttttcaggctctagaagcggcacagactttacactgactatctcatccctgcagcctgaggactttgctacatattattgtcaacaacattatactactccacccactttcggacagggcaccaaagtggagatcaaacgcaccggctccaccagtggaagcggtaagcctggctctggcgaaggctcagaagtgcaacttgtggagtctggaggggggctcgtccagcccggcggtagtctgaggctcagctgcgccgcatctggctttaatatcaaggacacatatatccactgggtacggcaagcaccaggtaagggactggagtgggtcgccagaatctaccccacaaacgggtacactcgctatgccgactcagtcaagggacgctttacaataagcgcagacacaagcaagaacaccgcttatctgcagatgaatagcttgcgggcggaggatacagctgtgtactactgcagcagatgggggggcgacggcttttacgctatggatgtgtggggccagggtactctggtgaccgtctcctccagatctggcggaggaggaggcgggcctctctcccctccgaccaatctccatctcgaagcaaaccctgacaccggggtcctcacggtttcatgggaaagaagtaccacgccggatataacgggttaccgcataacgaccacgccaacgaacgggcaacagggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggaggcggagaggtccagctgcagcagagtggtcctgaactggttaagcctggggcatcaatgaaaatctcctgtaaagcaagtggttattccttcaccggctatacaatgaactgggtgaagcagtctcacggaaaaaacctggaatggatggggctgattaatccgtataagggtgttagcacctacaaccagaaattcaaagataaggcaacactgactgtcgacaaaagctcctctaccgcttatatggaactgctgagcctgacatccgaggattctgccgtttattactgcgcgcgcagcggttattacggggattccgactggtactttgacgtgtggggccagggtaccacactgaccgttttcagcggcggtgggggatccggcggtgggggatctggcggtgggggaagtgatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagggcggaggccaccaccatcatcaccatcac

SEQ ID NO: 40 - protein, 607 aa, Artificial Sequence
  fusion protein
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSRSGGGGGGPLSPPTNLHLEANPDTGVLTVSWERSTTPDITGYRITTTPTNGQQGNSLEEVVHADQSSCTFDNLSPGLEYNVSVYTVKDDKESVPISDTIIPAVGGGGGGEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGHHHHHHH

SEQ ID NO: 41 - DNA, 1818 bp, Artificial Sequence
  fusion gene
gacattcaaatgacgcagtcaccctcttccctgtccgccagcgtgggggatcgcgtcacaatcacatgtcgcgcctctcaggatgtgaacaccgcggtggcttggtatcaacagaagccaggcaaagcacctaagctcctgatctactctgccagctttttgtacagcggcgtgccaagtaggttttcaggctctagaagcggcacagactttacactgactatctcatccctgcagcctgaggactttgctacatattattgtcaacaacattatactactccacccactttcggacagggcaccaaagtggagatcaaacgcaccggctccaccagtggaagcggtaagcctggctctggcgaaggctcagaagtgcaacttgtggagtctggaggggggctcgtccagcccggcggtagtctgaggctcagctgcgccgcatctggctttaatatcaaggacacatatatccactgggtacggcaagcaccaggtaagggactggagtgggtcgccagaatctaccccacaaacgggtacactcgctatgccgactcagtcaagggacgctttacaataagcgcagacacaagcaagaacaccgcttatctgcagatgaatagcttgcgggcggaggatacagctgtgtactactgcagcagatgggggggcgacggcttttacgctatggatgtgtggggccagggtactctggtgaccgtctcctccagatctggcggaggaggaggcggggtaagcgacgtcccccgagacctggaagtcgtggccgccacacccacttccctccttatctcttgggacgcacccgctgtcaccgttcggtattacaggatcacatatggagagacgggcggaaatagccccgtccaagagtttaccgtccctgggagtaaaagtacagccactataagtggcctcaaacctggtgttgattatacgatcaccgtctatgctgtgacggggagaggagatagtccagcgtcttcaaaacccattagcatcaattatcggaccggaggcgggggaggcggagaggtccagctgcagcagagtggtcctgaactggttaagcctggggcatcaatgaaaatctcctgtaaagcaagtggttattccttcaccggctatacaatgaactgggtgaagcagtctcacggaaaaaacctggaatggatggggctgattaatccgtataagggtgttagcacctacaaccagaaattcaaagataaggcaacactgactgtcgacaaaagctcctctaccgcttatatggaactgctgagcctgacatccgaggattctgccgtttattactgcgcgcgcagcggttattacggggattccgactggtactttgacgtgtggggccagggtaccacactgaccgttttcagcggcggtgggggatccggcggtgggggatctggcggtgggggaagtgatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagggcggaggccaccaccatcatcaccatcac

SEQ ID NO: 42 - protein, 606 aa, Artificial Sequence
  fusion protein
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSRSGGGGGGVSDVPRDLEVVAATPTSLLISWDAPAVTVRYYRITYGETGGNSPVQEFTVPGSKSTATISGLKPGVDYTITVYAVTGRGDSPASSKPISINYRTGGGGGGEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGHHHHHHH

SEQ ID NO: 43 - DNA, 1815 bp, Artificial Sequence
  fusion gene
gacatccagatgacacagactacatcctccctgtctgcctctctgggagacagagtcaccatcagttgcagggcaagtcaggacattagtaagtacctgaactggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaagatctggcggaggaggaggcgggcctctctcccctccgaccaatctccatctcgaagcaaaccctgacaccggggtcctcacggtttcatgggaaagaagtaccacgccggatataacgggttaccgcataacgaccacgccaacgaacgggcaacagggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggaggcggagaggtccagctgcagcagagtggtcctgaactggttaagcctggggcatcaatgaaaatctcctgtaaagcaagtggttattccttcaccggctatacaatgaactgggtgaagcagtctcacggaaaaaacctggaatggatggggctgattaatccgtataagggtgttagcacctacaaccagaaattcaaagataaggcaacactgactgtcgacaaaagctcctctaccgcttatatggaactgctgagcctgacatccgaggattctgccgtttattactgcgcgcgcagcggttattacggggattccgactggtactttgacgtgtggggccagggtaccacactgaccgttttcagcggcggtgggggatccggcggtgggggatctggcggtgggggaagtgatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagggcggaggccaccaccatcatcaccatcac

SEQ ID NO: 44 - protein, 605 aa, Artificial Sequence
  fusion protein
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSRSGGGGGGPLSPPTNLHLEANPDTGVLTVSWERSTTPDITGYRITTTPTNGQQGNSLEEVVHADQSSCTFDNLSPGLEYNVSVYTVKDDKESVPISDTIIPAVGGGGGGEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGHHHHHHH

SEQ ID NO: 45 - DNA, 1812 bp, Artificial Sequence
  fusion gene
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaagatctggcggaggaggaggcggggtaagcgacgtcccccgagacctggaagtcgtggccgccacacccacttccctccttatctcttgggacgcacccgctgtcaccgttcggtattacaggatcacatatggagagacgggcggaaatagccccgtccaagagtttaccgtccctgggagtaaaagtacagccactataagtggcctcaaacctggtgttgattatacgatcaccgtctatgctgtgacggggagaggagatagtccagcgtcttcaaaacccattagcatcaattatcggaccggaggcgggggaggcggagaggtccagctgcagcagagtggtcctgaactggttaagcctggggcatcaatgaaaatctcctgtaaagcaagtggttattccttcaccggctatacaatgaactgggtgaagcagtctcacggaaaaaacctggaatggatggggctgattaatccgtataagggtgttagcacctacaaccagaaattcaaagataaggcaacactgactgtcgacaaaagctcctctaccgcttatatggaactgctgagcctgacatccgaggattctgccgtttattactgcgcgcgcagcggttattacggggattccgactggtactttgacgtgtggggccagggtaccacactgaccgttttcagcggcggtgggggatccggcggtgggggatctggcggtgggggaagtgatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagggcggaggccaccaccatcatcaccatcac

SEQ ID NO: 46 - protein, 604 aa, Artificial Sequence
  fusion protein
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSRSGGGGGGVSDVPRDLEVVAATPTSLLISWDAPAVTVRYYRITYGETGGNSPVQEFTVPGSKSTATISGLKPGVDYTITVYAVTGRGDSPASSKPISINYRTGGGGGGEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGHHHHHHH

SEQ ID NO: 47 - DNA, 1731 bp, Artificial Sequence
  fusion gene
gacatccagatgacacagactacatcctccctgtctgcctctctgggagacagaatcaccatcagttgcagggcaagtcaggacattagtaagtacctgaactggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaagatctggcggaggaggaggcgggcctctctcccctccgaccaatctccatctcgaagcaaaccctgacaccggggtcctcacggtttcatgggaaagaagtaccacgccggatataacgggttaccgcataacgaccacgccaacgaacgggcaacagggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggaggcggagacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 48 - protein, 577 aa, Artificial Sequence
  fusion protein
DIQMTQTTSSLSASLGDRITISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSRSGGGGGGPLSPPTNLHLEANPDTGVLTVSWERSTTPDITGYRITTTPTNGQQGNSLEEVVHADQSSCTFDNLSPGLEYNVSVYTVKDDKESVPISDTIIPAVGGGGGGDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 2499 bp, Artificial Sequence
  fusion gene
gacatccagatgacacagactacatcctccctgtctgcctctctgggagacagagtcaccatcagttgcagggcaagtcaggacattagtaagtacctgaactggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaagatctggcggaggaggaggcgggcctctctcccctccgaccaatctccatctcgaagcaaaccctgacaccggggtcctcacggtttcatgggaaagaagtaccacgccggatataacgggttaccgcataacgaccacgccaacgaacgggcaacagggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggaggcggagacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggaagtgggggcggaggctctggtggtggcggagaggtccagctgcagcagagtggtcctgaactggttaagcctggggcatcaatgaaaatctcctgtaaagcaagtggttattccttcaccggctatacaatgaactgggtgaagcagtctcacggaaaaaacctggaatggatggggctgattaatccgtataagggtgttagcacctacaaccagaaattcaaagataaggcaacactgactgtcgacaaaagctcctctaccgcttatatggaactgctgagcctgacatccgaggattctgccgtttattactgcgcgcgcagcggttattacggggattccgactggtactttgacgtgtggggccagggtaccacactgaccgttttcagcggcggtgggggatccggcggtgggggatctggcggtgggggaagtgatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaag

SEQ ID NO: 50 - protein, 833 aa, Artificial Sequence
  fusion protein
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSRSGGGGGGPLSPPTNLHLEANPDTGVLTVSWERSTTPDITGYRITTTPTNGQQGNSLEEVVHADQSSCTFDNLSPGLEYNVSVYTVKDDKESVPISDTIIPAVGGGGGGDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGGGSGGGGEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 51 - DNA, 1749 bp, Artificial Sequence
  fusion gene
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgcggtggggaggtgacggcttctatgccatggactactggggccaaggaaccctggtcaccgtctcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacgggggaggaggcagtggcggaggaggaggcgggcctctctcccctccgaccaatctccatctcgaagcaaaccctgacaccggggtcctcacggtttcatgggaaagaagtaccacgccggatataacgggttaccgcataacgaccacgccaacgaacgggcaacagggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggaggcggagaggtccagctgcagcagagtggtcctgaactggttaagcctggggcatcaatgaaaatctcctgtaaagcaagtggttattccttcaccggctatacaatgaactgggtgaagcagtctcacggaaaaaacctggaatggatggggctgattaatccgtataagggtgttagcacctacaaccagaaattcaaagataaggcaacactgactgtcgacaaaagctcctctaccgcttatatggaactgctgagcctgacatccgaggattctgccgtttattactgcgcgcgcagcggttattacggggattccgactggtactttgacgtgtggggccagggtaccacactgaccgttttcagcggcggtgggggatccggcggtgggggatctggcggtgggggaagtgatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaag

SEQ ID NO: 52 - protein, 583 aa, Artificial Sequence
  fusion protein
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGGGGSGGGGGGPLSPPTNLHLEANPDTGVLTVSWERSTTPDITGYRITTTPTNGQQGNSLEEVVHADQSSCTFDNLSPGLEYNVSVYTVKDDKESVPISDTIIPAVGGGGGGEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 53 - DNA, 1722 bp, Artificial Sequence
  fusion gene
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaaggtggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgtgacaaaactcacgggggaggtggttctggcggaggaggaggcgggcctctctcccctccgaccaatctccatctcgaagcaaaccctgacaccggggtcctcacggtttcatgggaaagaagtaccacgccggatataacgggttaccgcataacgaccacgccaacgaacgggcaacagggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggaggcggagaggtccagctgcagcagagtggtcctgaactggttaagcctggggcatcaatgaaaatctcctgtaaagcaagtggttattccttcaccggctatacaatgaactgggtgaagcagtctcacggaaaaaacctggaatggatggggctgattaatccgtataagggtgttagcacctacaaccagaaattcaaagataaggcaacactgactgtcgacaaaagctcctctaccgcttatatggaactgctgagcctgacatccgaggattctgccgtttattactgcgcgcgcagcggttattacggggattccgactggtactttgacgtgtggggccagggtaccacactgaccgttttcagcggcggtgggggatccggcggtgggggatctggcggtgggggaagtgatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaag

SEQ ID NO: 54 - protein, 574 aa, Artificial Sequence
  fusion protein
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHGGGGSGGGGGGPLSPPTNLHLEANPDTGVLTVSWERSTTPDITGYRITTTPTNGQQGNSLEEVVHADQSSCTFDNLSPGLEYNVSVYTVKDDKESVPISDTIIPAVGGGGGGEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 55 - DNA, 1722 bp, Artificial Sequence
  fusion gene
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgacaaaactcacgggggaggtggttctggcggaggaggaggcgggcctctctcccctccgaccaatctccatctcgaagcaaaccctgacaccggggtcctcacggtttcatgggaaagaagtaccacgccggatataacgggttaccgcataacgaccacgccaacgaacgggcaacagggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggaggcggagacattcaaatgacgcagtcaccctcttccctgtccgccagcgtgggggatcgcgtcacaatcacatgtcgcgcctctcaggatgtgaacaccgcggtggcttggtatcaacagaagccaggcaaagcacctaagctcctgatctactctgccagctttttgtacagcggcgtgccaagtaggttttcaggctctagaagcggcacagactttacactgactatctcatccctgcagcctgaggactttgctacatattattgtcaacaacattatactactccacccactttcggacagggcaccaaagtggagatcaaacgcaccggctccaccagtggaagcggtaagcctggctctggcgaaggctcagaagtgcaacttgtggagtctggaggggggctcgtccagcccggcggtagtctgaggctcagctgcgccgcatctggctttaatatcaaggacacatatatccactgggtacggcaagcaccaggtaagggactggagtgggtcgccagaatctaccccacaaacgggtacactcgctatgccgactcagtcaagggacgctttacaataagcgcagacacaagcaagaacaccgcttatctgcagatgaatagcttgcgggcggaggatacagctgtgtactactgcagcagatgggggggcgacggcttttacgctatggatgtgtggggccagggtactctggtgaccgtctcctcc

SEQ ID NO: 56 - protein, 574 aa, Artificial Sequence
  fusion protein
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHGGGGSGGGGGGPLSPPTNLHLEANPDTGVLTVSWERSTTPDITGYRITTTPTNGQQGNSLEEVVHADQSSCTFDNLSPGLEYNVSVYTVKDDKESVPISDTIIPAVGGGGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 57 - DNA, 1719 bp, Artificial Sequence
  fusion gene
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgacaaaactcacgggggaggtggttctggcggaggaggaggcggggtaagcgacgtcccccgagacctggaagtcgtggccgccacacccacttccctccttatctcttgggacgcacccgctgtcaccgttcggtattacaggatcacatatggagagacgggcggaaatagccccgtccaagagtttaccgtccctgggagtaaaagtacagccactataagtggcctcaaacctggtgttgattatacgatcaccgtctatgctgtgacggggagaggagatagtccagcgtcttcaaaacccattagcatcaattatcggaccggaggcgggggaggcggagacattcaaatgacgcagtcaccctcttccctgtccgccagcgtgggggatcgcgtcacaatcacatgtcgcgcctctcaggatgtgaacaccgcggtggcttggtatcaacagaagccaggcaaagcacctaagctcctgatctactctgccagctttttgtacagcggcgtgccaagtaggttttcaggctctagaagcggcacagactttacactgactatctcatccctgcagcctgaggactttgctacatattattgtcaacaacattatactactccacccactttcggacagggcaccaaagtggagatcaaacgcaccggctccaccagtggaagcggtaagcctggctctggcgaaggctcagaagtgcaacttgtggagtctggaggggggctcgtccagcccggcggtagtctgaggctcagctgcgccgcatctggctttaatatcaaggacacatatatccactgggtacggcaagcaccaggtaagggactggagtgggtcgccagaatctaccccacaaacgggtacactcgctatgccgactcagtcaagggacgctttacaataagcgcagacacaagcaagaacaccgcttatctgcagatgaatagcttgcgggcggaggatacagctgtgtactactgcagcagatgggggggcgacggcttttacgctatggatgtgtggggccagggtactctggtgaccgtctcctcc

SEQ ID NO: 58 - protein, 573 aa, Artificial Sequence
  fusion protein
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHGGGGSGGGGGGVSDVPRDLEVVAATPTSLLISWDAPAVTVRYYRITYGETGGNSPVQEFTVPGSKSTATISGLKPGVDYTITVYAVTGRGDSPASSKPISINYRTGGGGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 59 - DNA, 1716 bp, Artificial Sequence
  fusion gene
gatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtgacaaaactcacgggggaggtggttctggcggaggaggaggcgggcctctctcccctccgaccaatctccatctcgaagcaaaccctgacaccggggtcctcacggtttcatgggaaagaagtaccacgccggatataacgggttaccgcataacgaccacgccaacgaacgggcaacagggcaactcacttgaggaggtcgttcatgccgatcaatcttcctgtacgtttgataatttgagtccgggacttgagtacaatgtcagtgtatataccgttaaagacgataaggagtcagtaccaatcagcgataccattattccggccgttggaggcgggggaggcggagacatccagatgacacagactacatcctccctgtctgcctctctgggagacagagtcaccatcagttgcagggcaagtcaggacattagtaagtacctgaactggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 60 - protein, 572 aa, Artificial Sequence
  fusion protein
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHGGGGSGGGGGGPLSPPTNLHLEANPDTGVLTVSWERSTTPDITGYRITTTPTNGQQGNSLEEVVHADQSSCTFDNLSPGLEYNVSVYTVKDDKESVPISDTIIPAVGGGGGGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 61 - DNA, 1746 bp, Artificial Sequence
  fusion gene
gaggtccagctgcagcagagtggtcctgaactggttaagcctggggcatcaatgaaaatctcctgtaaagcaagtggttattccttcaccggctatacaatgaactgggtgaagcagtctcacggaaaaaacctggaatggatggggctgattaatccgtataagggtgttagcacctacaaccagaaattcaaagataaggcaacactgactgtcgacaaaagctcctctaccgcttatatggaactgctgagcctgacatccgaggattctgccgtttattactgcgcgcgcagcggttattacggggattccgactggtactttgacgtgtggggccagggtaccacactgaccgttttcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacgggggaggtggttctggcggaggaggaggcggggtaagcgacgtcccccgagacctggaagtcgtggccgccacacccacttccctccttatctcttgggacgcacccgctgtcaccgttcggtattacaggatcacatatggagagacgggcggaaatagccccgtccaagagtttaccgtccctgggagtaaaagtacagccactataagtggcctcaaacctggtgttgattatacgatcaccgtctatgctgtgacggggagaggagatagtccagcgtcttcaaaacccattagcatcaattatcggaccggaggcgggggaggcggagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 62 - protein, 582 aa, Artificial Sequence
  fusion protein
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGGGGSGGGGGGVSDVPRDLEVVAATPTSLLISWDAPAVTVRYYRITYGETGGNSPVQEFTVPGSKSTATISGLKPGVDYTITVYAVTGRGDSPASSKPISINYRTGGGGGGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 63 - DNA, 732 bp, Artificial Sequence
  fusion gene
gaggtccagctgcagcagagtggtcctgaactggttaagcctggggcatcaatgaaaatctcctgtaaagcaagtggttattccttcaccggctatacaatgaactgggtgaagcagtctcacggaaaaaacctggaatggatggggctgattaatccgtataagggtgttagcacctacaaccagaaattcaaagataaggcaacactgactgtcgacaaaagctcctctaccgcttatatggaactgctgagcctgacatccgaggattctgccgtttattactgcgcgcgcagcggttattacggggattccgactggtactttgacgtgtggggccagggtaccacactgaccgttttcagcggcggtgggggatccggcggtgggggatctggcggtgggggaagtgatattcagatgactcagactaccagttcactgagcgcctccctgggcgatcgcgtgacaattagttgtcgtgcgtcacaggacatccggaactatctgaattggtaccagcagaagccggacggcacagtcaaactgctgatctattacactagccgtctgcattccggtgtgccctctaagttttctgggagtggatcaggcactgattatagtctgaccatttcaaacctggaacaggaagatatcgccacctacttctgtcagcaggggaatactctgccgtggactttcgccggaggaaccaaactggagattaag

SEQ ID NO: 64 - protein, 244 aa, Artificial Sequence
  fusion protein
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 65 - DNA, 732 bp, Artificial Sequence
  fusion gene
gacattcaaatgacgcagtcaccctcttccctgtccgccagcgtgggggatcgcgtcacaatcacatgtcgcgcctctcaggatgtgaacaccgcggtggcttggtatcaacagaagccaggcaaagcacctaagctcctgatctactctgccagctttttgtacagcggcgtgccaagtaggttttcaggctctagaagcggcacagactttacactgactatctcatccctgcagcctgaggactttgctacatattattgtcaacaacattatactactccacccactttcggacagggcaccaaagtggagatcaaacgcaccggctccaccagtggaagcggtaagcctggctctggcgaaggctcagaagtgcaacttgtggagtctggaggggggctcgtccagcccggcggtagtctgaggctcagctgcgccgcatctggctttaatatcaaggacacatatatccactgggtacggcaagcaccaggtaagggactggagtgggtcgccagaatctaccccacaaacgggtacactcgctatgccgactcagtcaagggacgctttacaataagcgcagacacaagcaagaacaccgcttatctgcagatgaatagcttgcgggcggaggatacagctgtgtactactgcagcagatgggggggcgacggcttttacgctatggatgtgtggggccagggtactctggtgaccgtctcctcc

SEQ ID NO: 66 - protein, 244 aa, Artificial Sequence
  fusion protein
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 67 - DNA, 726 bp, Artificial Sequence
  fusion gene
gacatccagatgacacagactacatcctccctgtctgcctctctgggagacagagtcaccatcagttgcagggcaagtcaggacattagtaagtacctgaactggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 68 - protein, 242 aa, Artificial Sequence
  fusion protein
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 69 - DNA, 285 bp, Artificial Sequence
  fusion gene
ccgccgaccaatctgcatctggaggccaatccagatacgggcgttctgaccgttagctgggaacgcagcaccaccccggatatcaccggctatcgcatcaccaccaccccgaccaatggccagcaaggcaccagtctggaagaagttgttcacgccgaccagagcagctgcaccttcgaaaatctgaatccgggtctggagtacaacgtgagcgtgtacacggtgaaagacgacaaagagagcgccccaatcagcgacaccgttgttccagccgttccgccgccg

SEQ ID NO: 70 - protein, 95 aa, Artificial Sequence
  fusion protein
PPTNLHLEANPDTGVLTVSWERSTTPDITGYRITTTPTNGQQGTSLEEVVHADQSSCTFENLNPGLEYNVSVYTVKDDKESAPISDTVVPAVPPP

SEQ ID NO: 71 - DNA, 282 bp, Artificial Sequence
  fusion gene
gtgagcgacatccctagagatttggaagtcatcgccagcacccccacctctctgctcatcagctgggagcctcccgctgtgagcgtgaggtactatagaatcacctacggcgagaccggcggcaactcccccgtgcaagagttcacagtgcccggcagcaagagcaccgccaccatcaacaacatcaagcccggcgccgactacaccatcacactgtacgctgtgaccggaagaggcgattcccccgccagctccaagcccgtctccatcaactataagacc

SEQ ID NO: 72 - protein, 94 aa, Artificial Sequence
  fusion protein
VSDIPRDLEVIASTPTSLLISWEPPAVSVRYYRITYGETGGNSPVQEFTVPGSKSTATINNIKPGADYTITLYAVTGRGDSPASSKPVSINYKT

SEQ ID NO: 73 - DNA, 999 bp, Artificial Sequence
  fusion gene
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccgccgaccaatctgcatctggaggccaatccagatacgggcgttctgaccgttagctgggaacgcagcaccaccccggatatcaccggctatcgcatcaccaccaccccgaccaatggccagcaaggcaccagtctggaagaagttgttcacgccgaccagagcagctgcaccttcgaaaatctgaatccgggtctggagtacaacgtgagcgtgtacacggtgaaagacgacaaagagagcgccccaatcagcgacaccgttgttccagccgttccgccgccgcaccaccatcatcaccatcac

SEQ ID NO: 74 - protein, 322 aa, Artificial Sequence
  fusion protein
PPTNLHLEANPDTGVLTVSWERSTTPDITGYRITTTPTNGQQGTSLEEVVHADQSSCTFENLNPGLEYNVSVYTVKDDKESAPISDTVVPAVPPPHHHHHHHMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 75 - DNA, 978 bp, Artificial Sequence
  fusion gene
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccgccgaccaatctgcatctggaggccaatccagatacgggcgttctgaccgttagctgggaacgcagcaccaccccggatatcaccggctatcgcatcaccaccaccccgaccaatggccagcaaggcaccagtctggaagaagttgttcacgccgaccagagcagctgcaccttcgaaaatctgaatccgggtctggagtacaacgtgagcgtgtacacggtgaaagacgacaaagagagcgccccaatcagcgacaccgttgttccagccgttccgccgccg

SEQ ID NO: 76 - protein, 315 aa, Artificial Sequence
  fusion protein
PPTNLHLEANPDTGVLTVSWERSTTPDITGYRITTTPTNGQQGTSLEEVVHADQSSCTFENLNPGLEYNVSVYTVKDDKESAPISDTVVPAVPPPMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 77 - DNA, 1113 bp, Artificial Sequence
  fusion gene
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccaccgaccaatctgcatctggaagccaatggcggtagcggtcgtcgcgccgttcgtaaccgtctgagcgaactgctgagcaagatcaacgacatgccgatcaccaacgaccagaagaagctcatgagcaacgacgtgctgaaatttgccgccgaagcggaaggtggtagtggcggtgtgctgacggttagctgggaacgcagcaccaccccggatatcacgggctaccgcatcaccaccaccccgaccaatggtcaacaaggcaccagtctggaggaagtggttcatgccgaccagagcagctgcaccttcgaaaatctgaacccgggtctggaatacaacgtgagcgtgtacaccgtgaaggacgacaaagagagcgccccgatcagcgataccgttgttccagccgttccaccgccg

SEQ ID NO: 78 - protein, 346 aa, Artificial Sequence
  fusion protein
RRAVRNRLSELLSKINDMPITNDQKKLMSNDVLKFAAEAEGGSGGVLTVSWERSTTPDITGYRITTTPTNGQQGTSLEEVVHADQSSCTFENLNPGLEYNVSVYTVKDDKESAPISDTVVPAVPPPMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 79 - DNA, 1116 bp, Artificial Sequence
  fusion gene
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccccgccgacgaatctgcatctggaagccaacccggataccggcgtgctgaccgttagctgggaacgcagcaccaccggtggtagcggtcgtcgtgcggttcgcaatcgtctgagcgaactgctgagcaagatcaacgacatgccgatcaccaacgaccagaagaagctgatgagcaacgacgtgctgaaattcgccgccgaagcggaaggtggtagcggcattaccggctaccgcatcaccacgaccccaaccaatggtcagcaaggcaccagtctggaagaggtggttcacgccgatcagagcagctgcaccttcgagaatctgaatccgggtctggaatacaacgtgagcgtgtacaccgtgaaggacgacaaagaaagcgcgccgatcagcgataccgtggttccagccgttccaccaccg

SEQ ID NO: 80 - protein, 361 aa, Artificial Sequence
  fusion protein
PPTNLHLEANPDTGVLTVSWERSTTGGSGRRAVRNRLSELLSKINDMPITNDQKKLMSNDVLKFAAEAEGGSGITGYRITTTPTNGQQGTSLEEVVHADQSSCTFENLNPGLEYNVSVYTVKDDKESAPISDTVVPAVPPPMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 81 - DNA, 306 bp, Artificial Sequence
  fusion gene
ccgccgaccaatctgcatctggaggccaatccagatacgggcgttctgaccgttagctgggaacgcagcaccaccccggatatcaccggctatcgcatcaccaccaccccgaccaatggccagcaaggcaccagtctggaagaagttgttcacgccgaccagagcagctgcaccttcgaaaatctgaatccgggtctggagtacaacgtgagcgtgtacacggtgaaagacgacaaagagagcgccccaatcagcgacaccgttgttccagccgttccgccgccgcaccaccatcatcaccatcac

SEQ ID NO: 82 - protein, 102 aa, Artificial Sequence
  fusion protein
PPTNLHLEANPDTGVLTVSWERSTTPDITGYRITTTPTNGQQGTSLEEVVHADQSSCTFENLNPGLEYNVSVYTVKDDKESAPISDTVVPAVPPPHHHHHHH

SEQ ID NO: 83 - DNA, 440 bp, Artificial Sequence
  fusion gene
caccgaccaatctgcatctggaagccaatggcggtagcggtcgtcgcgccgttcgtaaccgtctgagcgaactgctgagcaagatcaacgacatgccgatcaccaacgaccagaagaagctcatgagcaacgacgtgctgaaatttgccgccgaagcggaaggtggtagtggcggtgtgctgacggttagctgggaacgcagcaccaccccggatatcacgggctaccgcatcaccaccaccccgaccaatggtcaacaaggcaccagtctggaggaagtggttcatgccgaccagagcagctgcaccttcgaaaatctgaacccgggtctggaatacaacgtgagcgtgtacaccgtgaaggacgacaaagagagcgccccgatcagcgataccgttgttccagccgttccaccgccgcaccaccatcatcaccatcac

SEQ ID NO: 84 - protein, 146 aa, Artificial Sequence
  fusion protein
PTNLHLEANGGSGRRAVRNRLSELLSKINDMPITNDQKKLMSNDVLKFAAEAEGGSGGVLTVSWERSTTPDITGYRITTTPTNGQQGTSLEEVVHADQSSCTFENLNPGLEYNVSVYTVKDDKESAPISDTVVPAVPPPHHHHHHH

SEQ ID NO: 85 - DNA, 444 bp, Artificial Sequence
  fusion gene
ccgccgacgaatctgcatctggaagccaacccggataccggcgtgctgaccgttagctgggaacgcagcaccaccggtggtagcggtcgtcgtgcggttcgcaatcgtctgagcgaactgctgagcaagatcaacgacatgccgatcaccaacgaccagaagaagctgatgagcaacgacgtgctgaaattcgccgccgaagcggaaggtggtagcggcattaccggctaccgcatcaccacgaccccaaccaatggtcagcaaggcaccagtctggaagaggtggttcacgccgatcagagcagctgcaccttcgagaatctgaatccgggtctggaatacaacgtgagcgtgtacaccgtgaaggacgacaaagaaagcgcgccgatcagcgataccgtggttccagccgttccaccaccgcaccaccatcatcaccatcac

SEQ ID NO: 86 - protein, 148 aa, Artificial Sequence
  fusion protein
PPTNLHLEANPDTGVLTVSWERSTTGGSGRRAVRNRLSELLSKINDMPITNDQKKLMSNDVLKFAAEAEGGSGITGYRITTTPTNGQQGTSLEEVVHADQSSCTFENLNPGLEYNVSVYTVKDDKESAPISDTVVPAVPPPHHHHHHH

SEQ ID NO: 87 - DNA, 444 bp, Artificial Sequence
  fusion gene
ccgccaacgaatctgcatctggaagcgaatccggataccggtgtgctgaccgttagctgggaacgcagcaccaccccggatatcaccggctaccgcatcaccaccaccccaaccaatggtggtggtagtggtcgccgtgcggttcgcaatcgtctcagcgaactgctcagcaagatcaacgacatgccgatcacgaacgaccagaagaagctcatgagcaacgacgtgctgaaatttgccgccgaagcggaaggcggtagtggtggcacgagtctggaagaagtggtgcatgccgatcagagcagctgcacgttcgaaaatctgaatccgggtctggagtacaacgttagcgtgtacaccgtgaaggacgacaaagaaagcgcgccgatcagcgatacggttgttccagccgttccgccaccacaccaccatcatcaccatcac

SEQ ID NO: 88 - protein, 148 aa, Artificial Sequence
  fusion protein
PPTNLHLEANPDTGVLTVSWERSTTPDITGYRITTTPTNGGGSGRRAVRNRLSELLSKINDMPITNDQKKLMSNDVLKFAAEAEGGSGGTSLEEVVHADQSSCTFENLNPGLEYNVSVYTVKDDKESAPISDTVVPAVPPPHHHHHHH

SEQ ID NO: 89 - DNA, 444 bp, Artificial Sequence
  fusion gene
ccgccgaccaatctgcatctggaagccaatccggataccggcgttctgaccgtgagctgggaacgtagcacgaccccggatatcaccggctatcgcatcaccaccaccccgaccaacggtcagcaaggcaccagtctggaggaagtggttcatggtggtagcggtcgtcgcgcggttcgcaatcgtctgagcgagctgctgagcaagatcaacgacatgccgatcaccaacgatcagaagaagctgatgagcaacgatgtgctgaagttcgcggcggaagccgaaggcggtagtggtcagagcagttgcaccttcgagaatctgaacccgggtctggaatacaacgtgagcgtgtacaccgtgaaggacgacaaagaaagcgccccgatcagcgataccgttgtgccagccgtgccaccgccgcaccaccatcatcaccatcac

SEQ ID NO: 90 - protein, 148 aa, Artificial Sequence
  fusion protein
PPTNLHLEANPDTGVLTVSWERSTTPDITGYRITTTPTNGQQGTSLEEVVHGGSGRRAVRNRLSELLSKINDMPITNDQKKLMSNDVLKFAAEAEGGSGQSSCTFENLNPGLEYNVSVYTVKDDKESAPISDTVVPAVPPPHHHHHHH

SEQ ID NO: 91 - DNA, 444 bp, Artificial Sequence
  fusion gene
ccgccgaccaatctgcatctggaagccaatccagataccggtgtgctgaccgtgagctgggaacgtagcaccaccccggatatcaccggctaccgtatcaccaccaccccaacgaatggccaacaaggcaccagtctggaagaggtggtgcacgcggatcagagcagctgcaccttcgaaaatctgaatggtggcagcggtcgtcgtgccgttcgcaatcgcctcagcgaactgctgagcaagatcaacgacatgccgatcaccaacgaccagaagaagctgatgagcaacgacgtgctgaagttcgcggcggaagccgaaggtggtagcggtctggagtacaacgtgagcgtgtacaccgtgaaggatgataaggagagcgccccgattagcgataccgttgtgccagccgttccaccgccgcaccaccatcatcaccatcac

SEQ ID NO: 92 - protein, 148 aa, Artificial Sequence
  fusion protein
PPTNLHLEANPDTGVLTVSWERSTTPDITGYRITTTPTNGQQGTSLEEVVHADQSSCTFENLNGGSGRRAVRNRLSELLSKINDMPITNDQKKLMSNDVLKFAAEAEGGSGLEYNVSVYTVKDDKESAPISDTVVPAVPPPHHHHHHH

SEQ ID NO: 93 - DNA, 444 bp, Artificial Sequence
  fusion gene
ccaccaaccaatctgcatctggaggccaatccggataccggcgttctgaccgttagctgggaacgcagcaccaccccggatatcaccggctaccgcatcaccaccaccccaaccaacggtcagcaaggcaccagtctggaagaagtggtgcatgccgatcagagcagctgcaccttcgagaatctgaatccgggtctggagtacaatgtgagcgtgtacacggtgaaaggcggtagcggtcgtcgcgcggttcgcaatcgtctgagcgaactgctcagcaagatcaacgacatgccgatcaccaacgaccagaagaagctgatgagcaacgacgtgctgaagttcgccgcggaagccgaaggcggtagtggcaaggaaagcgccccgatcagcgatacggttgttccggccgttccgccgccgcaccaccatcatcaccatcac

SEQ ID NO: 94 - protein, 148 aa, Artificial Sequence
  fusion protein
PPTNLHLEANPDTGVLTVSWERSTTPDITGYRITTTPTNGQQGTSLEEVVHADQSSCTFENLNPGLEYNVSVYTVKGGSGRRAVRNRLSELLSKINDMPITNDQKKLMSNDVLKFAAEAEGGSGKESAPISDTVVPAVPPPHHHHHHH

SEQ ID NO: 95 - DNA, 303 bp, Artificial Sequence
  fusion gene
gtgagcgacatccctagagatttggaagtcatcgccagcacccccacctctctgctcatcagctgggagcctcccgctgtgagcgtgaggtactatagaatcacctacggcgagaccggcggcaactcccccgtgcaagagttcacagtgcccggcagcaagagcaccgccaccatcaacaacatcaagcccggcgccgactacaccatcacactgtacgctgtgaccggaagaggcgattcccccgccagctccaagcccgtctccatcaactataagacccaccaccatcatcaccatcac

SEQ ID NO: 96 - protein, 87 aa, Artificial Sequence
  fusion protein
VSDIPRDLEVIASTPTSLLISWEPPAVSVRYYRITYGETGGNSPVQEFTVPGSKSTATINNIKPGADYTITLYAVTGRGDSPASSKP

SEQ ID NO: 97 - DNA, 447 bp, Artificial Sequence
  fusion gene
gtgagcgacatccctagagatttggaagtcatcgccagcacccccacctctctgctcatcagctgggagcctcccgctgtgagcgtgaggtactatagaatcacctacggcgagaccggcggcaactcccccgtgcaagagttcacagtgcccggcagcaagagcaccgccaccatcaacaacatcaagcccggcgccgactacaccatcacactgtacgctgtgaccggaagaggcggcggcagcggaaggagagccgtgaggaacagactctccgagctgctgtccaaaatcaacgacatgcccatcaccaacgaccagaagaagctgatgtccaacgacgtgctgaagttcgctgccgaggccgaaggaggaagcggagattcccccgccagctccaagcccgtctccatcaactataagacccaccaccatcatcaccatcac

SEQ ID NO: 98 - protein, 87 aa, Artificial Sequence
  fusion protein
VSDIPRDLEVIASTPTSLLISWEPPAVSVRYYRITYGETGGNSPVQEFTVPGSKSTATINNIKPGADYTITLYAVTGRGGGSGRRAV

SEQ ID NO: 99 - DNA, 465 bp, Artificial Sequence
  fusion gene
caccatcatcatcatcatgaaaacctgtatttccagggtgtgagcgacatccctagagatttggaagtcatcgccagcacccccacctctctgctcatcagctgggagcctcccgctgtgagcgtgaggtactatagaatcacctacggcgagaccggcggcaactcccccgtgcaagagttcacagtgcccggcagcaagagcaccgccaccatcaacaacatcaagcccggcgccgactacaccatcacactgtacgctgtgaccggaagaggcggcggcagcggaaggagagccgtgaggaacagactctccgagctgctgtccaaaatcaacgacatgcccatcaccaacgaccagaagaagctgatgtccaacgacgtgctgaagttcgctgccgaggccgaaggaggaagcggagattcccccgccagctccaagcccgtctccatcaactataagacc

SEQ ID NO: 100 - protein, 155 aa, Artificial Sequence
  fusion protein
HHHHHHENLYFQGVSDIPRDLEVIASTPTSLLISWEPPAVSVRYYRITYGETGGNSPVQEFTVPGSKSTATINNIKPGADYTITLYAVTGRGGGSGRRAVRNRLSELLSKINDMPITNDQKKLMSNDVLKFAAEAEGGSGDSPASSKPVSINYKT

SEQ ID NO: 101 - DNA, 324 bp, Artificial Sequence
  fusion gene
atgcaccatcatcatcatcatgaaaacctgtatttccagggtgtgagcgacatccctagagatttggaagtcatcgccagcacccccacctctctgctcatcagctgggagcctcccgctgtgagcgtgaggtactatagaatcacctacggcgagaccggcggcaactcccccgtgcaagagttcacagtgcccggcagcaagagcaccgccaccatcaacaacatcaagcccggcgccgactacaccatcacactgtacgctgtgaccggaagaggcgattcccccgccagctccaagcccgtctccatcaactataagacc

SEQ ID NO: 102 - protein, 124 aa, Artificial Sequence
  fusion protein
DSPASSKPVSINYKTMHHHHHHENLYFQMGVSDIPRDLEVIASTPTSLLISWEPPAVSVRYYRITYGETGGNSPVQEFTVPGSKSTATINNIKPGADYTITLYAVTGRGDSPASSKPVSINYKT

SEQ ID NO: 103 - DNA, 519 bp, Artificial Sequence
  fusion gene
gtgagcgacatccctagagatttggaagtcatcgccagcacccccacctctctgctcatcagctgggagcctcccgctgtgagcgtgaggtactatagaatcacctacggcgagaccggcggcagtggtctggaacagttagaatctattatcaattttgaaaaactgaccgaatggacgagtggcggcagtggtaactcccccgtgcaagagttcacagtgcccggcagcaagagcaccgccaccatcaacaacatcaagcccggcgccgactacaccatcacactgtacgctgtgaccggaagaggcggcggcagcggaaggagagccgtgaggaacagactctccgagctgctgtccaaaatcaacgacatgcccatcaccaacgaccagaagaagctgatgtccaacgacgtgctgaagttcgctgccgaggccgaaggaggaagcggagattcccccgccagctccaagcccgtctccatcaactataagacccaccaccatcatcaccatcac

SEQ ID NO: 104 - protein, 129 aa, Artificial Sequence
  fusion protein
DSPASSKPVSINYKTHHHHHHHVSDIPRDLEVIASTPTSLLISWEPPAVSVRYYRITYGETGGSGLEQLESIINFEKLTEWTSGGSGNSPVQEFTVPGSKSTATINNIKPGADYTITLYAVTGRGGGSG

SEQ ID NO: 105 - DNA, 393 bp, Artificial Sequence
  fusion gene
caccatcatcatcatcatgaaaacctgtatttccagggtgtgagcgacatccctagagatttggaagtcatcgccagcacccccacctctctgctcatcagctgggagcctcccgctgtgagcgtgaggtactatagaatcacctacggcgagaccggcggcagtggtctggaacagttagaatctattatcaattttgaaaaactgaccgaatggacgagtggcggcagtggtaactcccccgtgcaagagttcacagtgcccggcagcaagagcaccgccaccatcaacaacatcaagcccggcgccgactacaccatcacactgtacgctgtgaccggaagaggcgattcccccgccagctccaagcccgtctccatcaactataagacc

SEQ ID NO: 106 - protein, 131 aa, Artificial Sequence
  fusion protein
HHHHHHENLYFQGVSDIPRDLEVIASTPTSLLISWEPPAVSVRYYRITYGETGGSGLEQLESIINFEKLTEWTSGGSGNSPVQEFTVPGSKSTATINNIKPGADYTITLYAVTGRGDSPASSKPVSINYKT

SEQ ID NO: 107 - DNA, 426 bp, Artificial Sequence
  fusion gene
gtaagcgacgtcccccgagacctggaagtcgtggccgccacacccggcggcagcggaaggagagccgtgaggaacagactctccgagctgctgtccaaaatcaacgacatgcccatcaccaacgaccagaagaagctgatgtccaacgacgtgctgaagttcgctgccgaggccgaaggaggaagcggaacttccctccttatctcttgggacgcacccgctgtcaccgttcggtattacaggatcacatatggagagacgggcggaaatagccccgtccaagagtttaccgtccctgggagtaaaagtacagccactataagtggcctcaaacctggtgttgattatacgatcaccgtctatgctgtgacggggagaggagatagtccagcgtcttcaaaacccattagcatcaattatcggacc

SEQ ID NO: 108 - protein, 142 aa, Artificial Sequence
  fusion protein
VSDVPRDLEVVAATPGGSGRRAVRNRLSELLSKINDMPITNDQKKLMSNDVLKFAAEAEGGSGTSLLISWDAPAVTVRYYRITYGETGGNSPVQEFTVPGSKSTATISGLKPGVDYTITVYAVTGRGDSPASSKPISINYRT

SEQ ID NO: 109 - DNA, 423 bp, Artificial Sequence
  fusion gene
gtaagcgacgtcccccgagacctggaagtcgtggccgccacacccacttccctccttatctcttgggacgcacccgctggcggcagcggaaggagagccgtgaggaacagactctccgagctgctgtccaaaatcaacgacatgcccatcaccaacgaccagaagaagctgatgtccaacgacgtgctgaagttcgctgccgaggccgaaggaggaagcggaaccgttcggtattacaggatcacatatggagagacgggcggaaatagccccgtccaagagtttaccgtccctgggagtaaaagtacagccactataagtggcctcaaacctggtgttgattatacgatcaccgtctatgctgtgacggggagaggagatagtccagcgtcttcaaaacccattagcatcaattatcggacc

SEQ ID NO: 110 - protein, 141 aa, Artificial Sequence
  fusion protein
VSDVPRDLEVVAATPTSLLISWDAPAGGSGRRAVRNRLSELLSKINDMPITNDQKKLMSNDVLKFAAEAEGGSGTVRYYRITYGETGGNSPVQEFTVPGSKSTATISGLKPGVDYTITVYAVTGRGDSPASSKPISINYRT

SEQ ID NO: 111 - DNA, 423 bp, Artificial Sequence
  fusion gene
gtaagcgacgtcccccgagacctggaagtcgtggccgccacacccacttccctccttatctcttgggacgcacccgctgtcaccgttcggtattacaggatcacatatggagagacgggcggcggcagcggaaggagagccgtgaggaacagactctccgagctgctgtccaaaatcaacgacatgcccatcaccaacgaccagaagaagctgatgtccaacgacgtgctgaagttcgctgccgaggccgaaggaggaagcggaaatagccccgtccaagagtttaccgtccctgggagtaaaagtacagccactataagtggcctcaaacctggtgttgattatacgatcaccgtctatgctgtgacggggagaggagatagtccagcgtcttcaaaacccattagcatcaattatcggacc

SEQ ID NO: 112 - protein, 141 aa, Artificial Sequence
  fusion protein
VSDVPRDLEVVAATPTSLLISWDAPAVTVRYYRITYGETGGGSGRRAVRNRLSELLSKINDMPITNDQKKLMSNDVLKFAAEAEGGSGNSPVQEFTVPGSKSTATISGLKPGVDYTITVYAVTGRGDSPASSKPISINYRT

SEQ ID NO: 113 - DNA, 426 bp, Artificial Sequence
  fusion gene
gtaagcgacgtcccccgagacctggaagtcgtggccgccacacccacttccctccttatctcttgggacgcacccgctgtcaccgttcggtattacaggatcacatatggagagacgggcggaaatagccccgtccaagagtttaccgtccctgggagtaaaagtacagccactataagtggcctcaaacctggtgttgattatacgatcaccgtctatgctgtgacggggagaggaggcggcagcggaaggagagccgtgaggaacagactctccgagctgctgtccaaaatcaacgacatgcccatcaccaacgaccagaagaagctgatgtccaacgacgtgctgaagttcgctgccgaggccgaaggaggaagcggagatagtccagcgtcttcaaaacccattagcatcaattatcggacc

SEQ ID NO: 114 - protein, 142 aa, Artificial Sequence
  fusion protein
VSDVPRDLEVVAATPTSLLISWDAPAVTVRYYRITYGETGGNSPVQEFTVPGSKSTATISGLKPGVDYTITVYAVTGRGGGSGRRAVRNRLSELLSKINDMPITNDQKKLMSNDVLKFAAEAEGGSGDSPASSKPISINYRT

SEQ ID NO: 115 - DNA, 120 bp, Artificial Sequence
  fusion gene
aggagagccgtgaggaacagactctccgagctgctgtccaaaatcaacgacatgcccatcaccaacgaccagaagaagctgatgtccaacgacgtgctgaagttcgctgccgaggccgaa

SEQ ID NO: 116 - protein, 40 aa, Artificial Sequence
  fusion protein
RRAVRNRLSELLSKINDMPITNDQKKLMSNDVLKFAAEAE

(19) United States
(12) Patent Application Publication
(10) Pub. No.: US 2021/0300995 A1
(43) Pub. Date: Sep. 30, 2021

(54) Long-acting fibronectin type III domain fusion protein

(71) Applicant: SHANGHAI YICHEN BIOMED CO. LTD, Shanghai (CN)

(72) Inventors: Feng WANG, Shanghai (CN); Huayang ZHENG, Shanghai (CN); Yuhan ZHANG, Shanghai (CN)

(21) Appl. No.: 17/261,026

(22) PCT Filed: Jul. 17, 2019
(86) PCT No.: PCT/CN2019/096357

§ 371 Date: Jan. 17, 2021

(30) Foreign Application Priority Data
Jul. 17, 2018 (CN) 201810784128.X

(51) Int. Cl. C07K 14/78; C07K 14/535; C07K 14/61; C07K 16/28; C07K 16/32; C12N 15/62; C12N 5/10

(52) U.S. Cl. CPC C07K 14/78; C07K 14/535; C07K 14/61; C07K 16/2809; C07K 16/32; C07K 2317/622; C12N 5/10; C07K 2319/00; C12N 2510/00; C12N 2511/00; C07K 2317/55; C12N 15/62

(57) ABSTRACT

The present application belongs to the field of biotechnology and pharmacy, and provides a fusion protein containing a fibronectin type III domain and preparation and application thereof. The structure of the fusion protein comprises a fibronectin type III domain and an insertion sequence, which can maintain the spatial conformation of an inserted protein or an active peptide, and can effectively protect the N-terminal and/or the C-terminal of a target protein, so that the target protein is not easily affected by enzymolysis, and thus obtaining a longer half-life in vivo.

### CROSS-REFERENCE TO RELATED PATENT APPLICATIONS

The present application is a National Stage of International Patent Application No. PCT/CN2019/096357, filed Jul. 17, 2019, and claims the priority of Chinese Patent Application No. 201810784128.X, filed on Jul. 17, 2018, the disclosures of which are incorporated herein by reference in their entirety.

### SEQUENCE LISTING

The instant application contains a Sequence Listing which has been submitted electronically in ASCII format and is hereby incorporated by reference in its entirety. Said ASCII copy is named PN146637_SEQ LIST.txt and is 225 kilobytes in size.

### TECHNICAL FIELD

The present application belongs to the field of biotechnology pharmacy, and particularly relates to a long-acting fibronectin III type domain fusion protein and preparation and use thereof.

### BACKGROUND

Since the first recombinant protein drug-recombinant human insulin was marketed in 1982, protein and polypeptide drugs have become one of the most important products in the field of modern biopharmaceuticals. More than 180 protein and polypeptide drugs are currently approved by the FDA for clinical use.

Compared with traditional small molecule chemical drugs, protein and polypeptide drugs have the advantages of strong specificity, low toxicity, small side effects, clear biological functions and so on. They have an irreplaceable role in the treatment of some diseases such as diabetes, hemophilia, rare diseases caused by protease deficiency and so on [2015 Recombinant protein drug research and development and patent analysis]. In addition to antibodies and Fc fusion proteins, most protein and polypeptide drugs generally have a molecular weight less than the upper limit of glomerular filtration elimination (60 kD), and they are readily metabolically eliminated by proteases or peptidases in the body, so the plasma half-life of such drugs is often short. For example, native glucagon-like peptide 1 (GLP-1) is easily digested and degraded by dipeptidyl peptidase 4 (DPP-4) with a plasma half-life of only 1-2 min. In addition, almost all protein and peptide drugs are administered parenterally. In order to achieve therapeutic effects, frequent or high-dose administration is required, which results in low patient compliance. Thus, protein modification techniques aimed at reducing the sensitivity of protein and peptide drugs to proteases and extending the plasma half-life have been used to improve the pharmacokinetics of these drugs.

Pharmacokinetic processes of a drug in vivo include absorption, distribution, metabolism and elimination. Protein and peptide drugs are absorbed by the lymphatic system and are widely distributed in the extracellular space of the central cavity. After being digested and metabolized by proteases and peptidases, macromolecular proteins are eliminated by the body through a receptor-mediated mechanism, while proteins and peptides with a molecular weight less than 60 kD are more easily eliminated by glomerular filtration. For small proteins and polypeptides, currently clinically used strategies to prolong their plasma half-life can be divided into two major categories: 1) fusion of a protein or polypeptide with a native long half-life protein or protein domain such as Fc, human serum albumin (HSA) and the like, facilitating protein recycling in vivo through an FcR-mediated mechanism; 2) Fusion of a protein or polypeptide with an inert polypeptide such as poly-ethylene glycol (PEG), XTEN (also known as recombinant PEG, rPEG), and the like, increasing the apparent molecular weight of the protein and preventing its elimination by glomerular filtration.

In plasma proteins, IgG, HSA and transferrin have longer half-lives than other proteins. The relative molecular weights of these three proteins were all greater than the renal filtration threshold. IgG and HSA could also be recycled in vivo through the mechanism mediated by neonatal Fc receptor (FcRn), while transferrin can prolong the residence time in vivo through clathrin-dependent transferrin receptors, so they all had a long half-life in vivo. Fusion of proteins, polypeptides and these proteins tend to significantly increase half-life.

As mentioned above, human immunoglobulins IgG1, IgG2, and IgG4 subtypes can be recycled in the body through pH-dependent FcRn mediation, so the plasma half-life of these IgG subtypes can reach 3-4 weeks. The interaction site of the immunoglobulin and the FcRn is an Fc region, and under an acidic environment, the IgG is combined with the FcRn on the cell membrane, avoiding the degradation of lysosomes. In a neutral environment, it is released into the blood again. Fc fusion is currently the most studied and fastest-developing protein fusion technology. The Fc fusion protein not only can improve the half-life, retain the biological activity of the fusion protein, but also has the antibody activity of Fc. In addition, the Fc fusion technology also has other advantages—the combination with the FcRn receptor provides a new way for the fusion protein to be absorbed in the body, e.g., EPO, follicle stimulating hormone and interferon-α/β can pass through endothelial cells after the fusion with Fc, so administration can be made by inhalation through the upper respiratory tract [Kuo T T, Baker K, Yoshida M, et al. Neonatal Fc receptor: from immunity to therapeutics. J Clin Immunol 2010; 30(6):777-789]. Since the first Fc fusion drug Enbrel (etanercept) was approved by the FDA in 1998 to May 2015, 11 Fc fusion recombinant proteins have been approved for clinical use. The plasma half-life of these Fc-fused recombinant proteins is much longer than that of the unfused proteins, e.g., Fc-fused factor IX (Alprolix, eftrenonacog-alpha; factor IX-Fc, approximately 98 kD, has a plasma half-life of 57-83 hours, more than 3 times that of factor IX alone (half-life 18 hours) [Shapiro A D, Ragni M V, Valentino L A, et al. Recombinant factor IX-Fc fusion protein (rFIX-Fc) demonstrates safety and prolonged activity in a phase 1/2a study in hemophilia B patients. Blood 2012; 119:666-72][Powell J S, Pasi K J, Ragni M V, et al. Phase 3 study of recombinant factor IX Fc fusion protein in hemophilia B. N Engl J Med 2013; 369:2313-23]. The half-life of native GLP-1 in vivo is only 2 minutes. The plasma half-life of Trulicity (dulaglutide; GLP-1-Fc fusion protein) developed by Eli Lilly is 4-5 days. In clinical use, it only needs to be administered once a week, which greatly improves patient medication compliance. The drug was approved for marketing in 2014 [Glaesner W, Vick A M, Millican R, et al. Engineering and characterization of the long-acting glucagon-like peptide-1 anaglogue LY2189265, an Fc fusion protein. Diabetes Metab Res Rev. 2010; 26:287-96.] At present, Fc mainly forms fusion proteins with the extramembrane region of the receptor and polypeptides. Other types of Fc fusion proteins are rarely developed in the later stages of clinical development. Due to structural limitations, Fc is generally fused to the C-terminal of the target protein and forms a dimer. This kind of fusion method often leads to reduced protein stability and significantly weakened biological activity due to conformational interference and steric hindrance. In addition, Fc mainly uses IgG1 subtype Fc, which may have unnecessary side effects due to ADCC and CDC activities.

HSA is the protein with the highest content in plasma, which plays an important role in maintaining plasma pH, transporting metabolites and fatty acids, and stabilizing blood pressure. Similar to IgG, HSA can be recycled in vivo through pH-dependent FcRn mediation. Its molecular weight (MW=66.5 Kd) just exceeds the upper limit of glomerular filtration elimination, and it is strongly negatively charged and will be rejected by the glomerular basement membrane, so its plasma half-life is as long as 19 days. Due to its good water solubility, no immunogenicity, wide tissue distribution, no enzyme activity, and easy aggregation in tumor and inflammatory tissues, HSA is used as a fusion partner. Linking it to protein drugs with a short half-life can not only increase the relative molecular mass and hydration radius of the drug molecule, but also extend the half-life of the drug by using the FcRn-mediated recycling mechanism. The first HSA-fused drug was Tanzeum (GLP 1-HSA) marketed by GSK in 2014, which extended the half-life of native GLP-1 from 1-2 days to 4-7 days. Similar to Trulicity of Eli Lilly, Tanzeum only needs to be administered once a week, greatly improving patient medication compliance. According to the PC-DACTM technology of ConjuChem LLC, peptide drugs are combined with HSA before administration, which extends the half-life of GLP-1 by more than 6000 times [Bosse D, Praus M, Kiessling P, et al. Phase I comparability of recombinant human albumin and human serum albumin. J Clin Pharmacol 2005, 45(1): 57-67]. Balugrastim (GCSF-HSA) from Teva has now completed a phase III clinical trial with a half-life that is more than 7-fold higher than that of G-CSF alone. CSL654 (rFIX-FP) and CSL689 (rFVIIa-FP) of CSL, Albuferon (IFN-a2b-HAS) of Novartis, MM-111 (Her3-HSA-Her2 bispecific antibody fusion protein) of Merrimack, etc. are all in the clinical trial stage. Some problems have also been discovered during the development of protein drugs fused with HSA. For example, the fusion of the target protein and HSA may lead to a decrease in the activity of the target protein and lower medicinal value; and the fusion protein is also prone to degradation and polymerization during fermentation, purification and storage.

PEG is a highly flexible, non-charged, and almost non-immunogenic hydrophilic polymer. It has been recognized by the FDA as GRAS (generally recognized as safe) and has been approved to extend the half-life of protein or peptide drugs for more than 20 years. At present, 12 PEGylated drugs have been successfully applied to the clinic by the FDA, such as PegIntron® (PEGylated interferon alfa-2b), Pegasys® (PEGylated IFN-a2a) for the treatment of hepatitis B, Neulasta® (pegfilgrastim, PEG-conjugated granulocyte colony stimulating factor) for the treatment of chemotherapy-induced neutropenia, and Mycera (a PEGylated form of erythropoietin-b) for the treatment of anemia in patients with chronic kidney disease, [Turecek P L, Bossard M J, Schoetens F, Ivens I A. PEGylation of Biopharmaceuticals: A Review of Chemistry and Nonclinical Safety Information of Approved Drugs. J Pharm Sci. 2016 February; 105(2):460-75]. PEG modification increases the water solubility and apparent molecular weight of the protein, reduces the filtration and elimination of the protein by the kidney, and protects the protein from enzymatic hydrolysis. Therefore, the frequency of administration can be reduced to once a week. Due to the polydispersity and heterogeneity of PEG, PEG-modified protein drugs need to be further modified on the protein, which poses high challenges for quality research, process control, and product quality control. For proteins such as cytokines and growth hormones with relatively small molecular weights, the steric hindrance caused by PEGylation hinders the binding to the corresponding receptors, resulting in decreased apparent activity. PEG is non-degradable in vivo and would accumulate in the kidney after long-term high-dose injection of PEG-interferon (PEG-IFNa 2a) (Conover C D et al. Artificial Organs 197; 21:36-378; Bendele A et al. Toxicol Sci 1998; 42:152-157). Non-clinical toxicity studies have shown that 5 of the 12 PEG-modified drugs approved by the FDA can lead to the formation of cell vacuoles, and the formation of vacuoles is related to PEG. Two of them (Somavert® and Krystexxa®) are coupled to multiple small molecular weight PEGs (5 kD and 10 kD, respectively), and the other three (Omontys®, Macugen® and Cimzia®) are coupled to a single PEG molecule of 40 kD [Ivens I A, Achanzar W, Baumann A, et al. PEGylated biopharmaceuticals: current experience and considerations for nonclinical development. Toxicol Pathol. 2015; 43(7):959-983]. In addition, PEG is expensive and requires chemical coupling with proteins and subsequent purification. Therefore, the application of PEG is still greatly restricted from the perspective of drug design. [Fee C J, Van Alstine J N. Purification of PEGylated proteins. In: Janson J-C, editor. Protein purification: principles, high resolution methods, and applications. 3rd ed. New York: Wiley; 2011. p. 339-62.].

The PEG mimetic XTEN is an in vivo degradable, non-immunogenic, amorphous polymer composed of six hydrophilic, structurally stable amino acids (alanine (Ala), glutamic acid (Glu), glycine (Gly), proline (Pro), serine (Ser), threonine (Thr)) developed by Amunix. Studies have shown that XTEN fusion of 288aa (32 kD) to 1008 aa (111 kD) can prolong the half-life of exenatide from 50-125 fold in different animal models (mouse, rat, monkey) [Schellenberger V, Wang C W, Geething N C, et al. A recombinant polypeptide extends the in vivo half-life of peptides and proteins in a tunable manner. Nat Biotechnol. 2009; 27:1186-90]. XTENylated exenatide (VRS-859) has entered clinical stage I for blood glucose control in type 2 diabetic patients; the half-life of VRS-859 was 65-71 times longer than exenatide in mouse and rat models, and from 30 min to 60 hrs in monkeys [Schellenberger V, Wang C W, Geething N C, et al. A recombinant polypeptide extends the in vivo half-life of peptides and proteins in a tunable manner. Nat Biotechnol. 2009; 27:1186-90]. Another XTEN fusion protein entering clinical phase III is VRS-317 (XTENylated hGH), and studies have shown that VRS-317 has a human body half-life of 131 h [Yuen K C J, Conway G S, Popovic V, et al. A long-acting human growth hormone with delayed clearance (VRS-317): results of a double-blind, placebo-controlled, single ascending dose study in growth hormone-deficient adults. J Clin Endrocrin Metab. 2013; 98:2595-.], much higher than that of PEGylated rhGH and CTP-hGH fusion protein MO603D-4023 [Strohl W R. Fusion Proteins for Half-Life Extension of Biologics as a Strategy to Make Biobetters. BioDrugs. 2015 August; 29(4):215-39]. Similar to PEG, for proteins with relatively small molecular weight, such as cytokines, and growth hormone, the steric effect caused by XTENylation may result in a decrease in the biological activity of the protein; in addition, XTEN is a highly water-soluble polymer, which often leads to the increase of viscosity of fusion proteins, and is laborious to separate and purify, and it is difficult to make a preparation. There is currently no officially approved fusion protein for clinical use. Therefore, its safety needs to be further evaluated.

In this technical field, there is also a need to develop more fusion partner molecules to generate fusion proteins with protein or peptide drugs.

### SUMMARY

The inventors have found that the fibronectin type III domain is an excellent fusion partner molecule. After fused with a variety of proteins or peptides, it can maintain or even enhance the activity and stability of the inserted fused protein or peptide, and can significantly increase the half-life of the protein or peptide in vivo; in addition, As an endogenous protein, the extremely low immunogenicity of the fibronectin makes itself an ideal carrier for long-acting fusion proteins and subunit vaccines. The present application has thus been completed.

Specifically, this application provides the following solutions:

1. A fibronectin type III domain fusion protein, comprising:

a fibronectin type III domain;

one or more linkers; and

a first physiologically active peptide.

2. The fibronectin type III domain fusion protein of embodiment 1, wherein the first physiologically active peptide is inserted within the fibronectin type III domain.

3. The fibronectin type III domain fusion protein of embodiment 2, wherein the first physiologically active peptide is inserted within a flexible loop formed between two adjacent β chains, such as selected from the group consisting of AB loop, BC loop, CD loop, DE loop, EF loop or FG loop, of the fibronectin type III domain.

4. The fibronectin type III domain fusion protein of any one of embodiments 2-3, further comprising a second physiologically active peptide.

5. The fibronectin type III domain fusion protein of embodiment 4, wherein the second physiologically active peptide is inserted within a flexible loop formed between two adjacent β chains, such as selected from the group consisting of AB loop, BC loop, CD loop, DE loop, EF loop or FG loop, of the fibronectin type III domain by a linker, and the second physiologically active peptide and the first physiologically active peptide are inserted at different positions of the fibronectin type III domain.

6. The fibronectin type III domain fusion protein of embodiment 4, wherein the second physiologically active peptide is linked to the N-terminal or the C-terminal of the fibronectin type III domain by a linker.

7. The fibronectin type III domain fusion protein of embodiment 1, wherein the first physiologically active peptide is linked to the N-terminal or the C-terminal of the fibronectin type III domain by a linker.

8. The fibronectin type III domain fusion protein of embodiment 7, further comprising a second physiologically active peptide.

9. The fibronectin type III domain fusion protein of embodiment 8, wherein the second physiologically active peptide is inserted within a flexible loop formed between two adjacent β chains, such as selected from the group consisting of AB loop, BC loop, CD loop, DE loop, EF loop or FG loop, of the fibronectin type III domain by a linker.

10. The fibronectin type III domain fusion protein of embodiment 8, wherein the second physiologically active peptide is inserted at the N-terminal or the C-terminal of the fibronectin type III domain fusion protein by a linker, and the second physiologically active peptide and the first physiologically active peptide are connected to opposite terminus of the fibronectin type III domain.

11. The fibronectin type III domain fusion protein of any one of embodiments 1-10, wherein the fibronectin type III domain is a fibronectin 7^{th} type III domain (FN7).

12. The fibronectin type III domain fusion protein of embodiment 11, wherein the FN7 is human FN7, in particular FN7 as shown in SEQ ID NO: 2.

13. The fibronectin type III domain fusion protein of embodiment 11, wherein the FN7 is mouse FN7, in particular FN7 as shown in SEQ ID NO: 70.

14. The fibronectin type III domain fusion protein of any one of embodiments 1-10, wherein the fibronectin type III domain is a fibronectin 10^{th} type III domain (FN10).

15. The fibronectin type III domain fusion protein of embodiment 14, wherein the FN10 is human FN10, in particular FN10 as shown in SEQ ID NO: 4.

16. The fibronectin type III domain fusion protein of embodiment 14, wherein the FN10 is mouse FN7, in particular FN10 as shown in SEQ ID NO: 72.

17. The fibronectin type III domain fusion protein of any one of embodiments 1-16, wherein at least one linker in the fusion protein is a flexible peptide, i.e., a polypeptide having a flexible structure.

18. The fibronectin type III domain fusion protein of embodiment 17, wherein each linker in the fusion protein is a flexible peptide.

19. The fibronectin type III domain fusion protein of embodiment 17 or 18, wherein the flexible peptide consists of small molecular weight polar amino acids such as glycine (Gly), serine (Ser), threonine (Thr), alanine (Ala), glutamic acid (Glu) or phenylalanine (Phe).

20. The fibronectin type III domain fusion protein of embodiment 19, wherein the flexible peptide is selected from the group consisting of: (G4S)ₙ, wherein n=1, 2, 3, 4 or 5; (Gly)₈, (Gly)₆, GGGSGGGGS, GGGGSGGGS, GSAGSAAGSGEF, KESGSVSSEQLAQFRSLD or EGKSSGSGSESKST.

21. The fibronectin type III domain fusion protein of any one of embodiments 1-16, wherein at least one linker in the fusion protein is a rigid peptide, preferably the rigid peptide consists of α-helices.

22. The fibronectin type III domain fusion protein of embodiment 21, wherein each linker in the fusion protein is a rigid peptide consisting of α-helices.

23. The fibronectin type III domain fusion protein of embodiment 21 or 22, wherein an amino acid sequence of the rigid peptide consisting of α-helices is selected from the group consisting of: (EAAAK)ₙ, wherein n=1, 2, 3, 4, or 5; and A (EAAAK)ₙA (n=2-5).

24. The fibronectin type III domain fusion protein of any one of embodiments 1-23, wherein the linker substitutes or does not substitute one or more amino acid residues within the AB loop, BC loop, CD loop, DE loop, EF loop, or FG loop of the fibronectin type III domain.

25. The fibronectin type III domain fusion protein of any one of embodiments 1-23, wherein the linker does not substitute any amino acid residue at the N-terminal or the C-terminal of the fibronectin type III domain.

26. The fibronectin type III domain fusion protein of any one of embodiments 1-25, wherein the first physiologically active peptide is selected from: the group consisting of a hormone, a cytokine, a vaccine antigen, an antigen protein, an interleukin, an interleukin-fusion protein, an enzyme, an antibody, a growth factor, a transcription regulatory factor, a coagulation factor, a structural protein, a ligand protein and a receptor, a receptor antagonist, a cell surface antigen, an antibody or an antigen-binding fragment thereof and a toxic protein.

27. The fibronectin type III domain fusion protein of embodiment 26, wherein the first physiologically active peptide is selected from the group consisting of: human growth factor, a colony stimulating factor, a viral-derived antigenic protein, a Fc, growth hormone releasing peptide, an interferon, an interferon receptor, a monoclonal antibody, a polyclonal antibody and an antibody fragment, glucagon-like peptide, a G protein-coupled receptor, an interleukin, an interleukin receptor, an enzyme, an interleukin binding protein, a cytokine binding protein, a macrophage activating factor, a B cytokine, a T cytokine, protein A, an allergy inhibitor, a cell necrosis glycoprotein, an immunotoxin, a lymphotoxin, a tumor necrosis factor, a tumor suppressor, a transforming growth factor, α-1 antitrypsin, albumin, α-lactalbumin, apolipoprotein-E, erythropoietin, a highly glycosylated erythropoietin, an angiopoietin, hemoglobin, thrombin, a thrombin receptor activating peptide, thrombomodulin, factor VII, factor VIIa, factor VIII, factor IX, factor XIII, a plasminogen activator, a fibrin-binding peptide, urokinase, streptokinase, hirudin, protein C, C-reactive protein, a renin inhibitor, a collagenase inhibitor, a superoxide dismutase, leptin, a platelet-derived growth factor, an epithelial growth factor, an epidermal growth factor, an angiostatin, an angiotensin, a bone growth factor, a bone stimulating protein, calcitonin, insulin, atrial peptide hormone, cartilage-inducing factor, elcatonin, a connective tissue activating factor, a tissue factor pathway inhibitor, follicle-stimulating hormone, luteinizing hormone, luteinizing hormone releasing hormone, a nerve growth factor, parathyroid hormone, relaxin, secretin, a stomatomedin, an insulin-like growth factor, an adreno cortical hormone, glucagon, cholecystokinin, pancreatic polypeptide, gastrin releasing peptide, a corticotropin releasing factor, thyroid stimulating hormone, an autocrine motility factor, lactoferrin, tubocurarine, a receptor, a receptor antagonist or a cell surface antigen and the like.

28. The fibronectin type III domain fusion protein of embodiment 27, wherein the first physiologically active) peptide is selected from the group consisting of: human growth factor, human granulocyte colony stimulating factor, RSV F protein, OVA, a Fc, a Fab heavy chain, a Fab light chain or a scFv.

29. The fibronectin type III domain fusion protein of any one of embodiments 1-28, wherein the second physiologically active peptide is selected from the group consisting of: a hormone, a cytokine, a vaccine antigen, an antigen protein, an enzyme, a growth factor, a transcription regulatory factor, a coagulation factor a structural protein, a ligand protein and a receptor, an antibody or an antigen-binding fragment thereof, and a toxic protein.

30. The fibronectin type III domain fusion protein of embodiment 29, wherein the second physiologically active peptide is selected from the group consisting of: human growth factors, a colony stimulating factor, a viral-derived antigenic protein, a Fc, growth hormone releasing peptide, an interferon, an interferon receptor, glucagon-like peptide, a G protein-coupled receptor, an interleukins, an interleukin receptor, an enzyme, an interleukin binding protein, a cytokine binding protein, a macrophage activating factor, a B cytokine, a T cytokine, protein A, an allergy inhibitor, a cell necrosis glycoprotein, an immunotoxin, a lymphotoxin, a tumor necrosis factor, tumor suppressor, a transforming growth factor, α-1 antitrypsin, albumin, α-lactalbumin, apolipoprotein-E, erythropoietin, a highly glycosylated erythropoietin, an angiopoietin, hemoglobin, thrombin, a thrombin receptor activating peptide, thrombomodulin, factor VII, factor VIIa, factor VIII, factor IX, factor XIII, a plasminogen activator, a fibrin-binding peptide, urokinase, streptokinase, hirudin, protein C, C-reactive protein, a renin inhibitor, a collagenase inhibitor, a superoxide dismutase, leptin, a platelet-derived growth factor, an epithelial growth factor, an epidermal growth factor, an angiostatin, an angiotensin, a bone growth factor, a bone stimulating protein, calcitonin, insulin, atrial peptide hormone, cartilage-inducing factor, elcatonin, a connective tissue activating factor, a tissue factor pathway inhibitor, follicle-stimulating hormone, luteinizing hormone, luteinizing hormone releasing hormone, a nerve growth factor, parathyroid hormone, relaxin, secretin, a stomatomedin, an insulin-like growth factor, an adreno cortical hormone, glucagon, cholecystokinin, pancreatic polypeptide, gastrin releasing peptide, a corticotropin releasing factor, thyroid stimulating hormone, an autocrine motility factor, lactoferrin, tubocurarine, a receptor, a receptor antagonist or a cell surface antigen, a virus-derived vaccine antigen, a monoclonal antibody, a polyclonal antibody and an antibody fragment.

31. The fibronectin type III domain fusion protein of embodiment 29, wherein the second physiologically active peptide is selected from the group consisting of: human growth factor, human granulocyte colony stimulating factor, RSV F protein, OVA, a Fab heavy chain, a Fab light chain or a scFv.

32. The fibronectin type III domain fusion protein of embodiment 1, comprising an amino acid sequence shown in any one of SEQ ID NOs: 10, 12, 14, 16, 18, 20, 22, 24, 26, 28, 30, 32, 34, 36, 38, 40, 42, 44, 46, 48, 50, 52, 54, 56, 58, 60, 62, 74, 76, 78, 80, 82, 84, 86, 88, 90, 92, 94, 96, 98, 100, 104, 106, 108, 110, 112, and 114.

33. A polynucleotide encoding a fibronectin type III domain fusion protein of any one of embodiments 1-32, preferably comprising a nucleotide sequence selected from the group consisting of: SEQ ID NOs: 9, 11, 13, 15, 17, 19, 21, 23, 25, 27, 29, 31, 33, 35, 37, 39, 41, 43, 45, 47, 49, 51, 53, 55, 57, 59, 61, 73, 75, 77, 79, 81, 83, 85, 87, 89, 91, 93, 95, 97, 99, 103, 105, 107, 109, 111, and 113.

34. An expression vector comprising the polynucleotide of embodiment 33.

35. A host cell comprising the expression vector of embodiment 34.

36. The host cell of embodiment 35, wherein the host cell is a mammalian host cell transiently transfected with the expression vector of embodiment 30.

37. A method for preparing the fibronectin type III domain fusion protein of any one of embodiments 1-32, comprising: culturing the mammalian host cell of embodiment 35 under conditions that permit expression of the fibronectin type III domain fusion protein; and collecting the fibronectin type III domain fusion proteins secreted from culture supernatants.

38. Use of the fibronectin type III domain fusion protein of any one of embodiments 1-32 for the preparation of a medicament.

39. Use of a fibronectin type III domain selected from any one or more of: 1) increasing the activity of a physiologically active peptide; 2) improving the stability of the physiologically active peptide; 3) prolonging the plasma half-life of the physiologically active peptide; 4) serving as a carrier for a vaccine antigen protein or a polypeptide.

40. The use of embodiment 39, wherein the fibronectin type III domain is:

(a) a fibronectin 7^{th} type III domain (FN7), wherein the FN is human FN7, in particular a FN7 having the sequence shown in SEQ ID NO: 2; alternatively, the FN is mouse FN7, in particular a FN7 with the sequence shown in SEQ ID NO: 70; or,

(b) a fibronectin 10^{th} type III domain (FN10), wherein the FN10 is human FN10, in particular a FN10 having the sequence shown in SEQ ID NO: 4; alternatively, the FN10 is mouse FN10, in particular a FN10 having the sequence shown in SEQ ID NO: 72.

41. The use of embodiment 39, wherein the use is achieved by preparing the fibronectin type III domain and the physiologically active peptide as a fusion protein as described in any one of embodiments 1-32.

42. The use of any one of embodiments 38-41, wherein the physiologically active peptide is selected from the group consisting of: a hormone, a cytokine, a vaccine antigen, an antigen protein, an enzyme, a growth factor, a transcription regulatory factor, a coagulation factor, a structural protein, a ligand protein and a receptor, an antibody or an antigen-binding fragment thereof and a toxic protein; preferably, the second physiologically active peptide is selected from the group consisting of: human growth factor, human granulocyte colony stimulating factor, RSVF protein, OVA protein, a Fc, a Fab heavy chain, a Fab light chain or a scFV.

Beneficial Effects:

According to the present application, the fibronectin III type domain is used as a carrier of the fusion protein, which can maintain the physiological activity of the inserted and/or fused physiologically active peptide, and significantly increase the in vivo half-life of the physiologically active peptide. Due to its low immunogenicity, it is an ideal carrier for preparing long-acting fusion proteins and subunit protein vaccines.

### BRIEF DESCRIPTION OF THE DRAWINGS

FIG. 1 is a schematic representation of a fibronectin type III domain in which arrows indicate positions at which foreign proteins or polypeptides may be inserted.

FIG. 2 is a schematic representation of a fibronectin type III domain inserted with a foreign protein or polypeptide.

FIG. 3 is a schematic representation of the sequence alignment and secondary structures of different fibronectin type III domain in mice and humans. Arrows indicate the β sheet.

FIGS. 4A-E are SDS-PAGE gel images of fibronectin type III domain fusion proteins. M: protein marker. In FIG. 4A, lane 1: FN10, lane 2: FN10-3a-GCSF, lane 3: FN10-3g-GCSF, lane 4: FN10-6g-GCSF, lane 5: FN10-6a-GCSF, lane 6: FN7-3g-GCSF-Fc, and lane 7: FN7-3a-hGH-Fc. In FIG. 4B, lane 1: ScFv3-FN7-ScFv1, lane 2: ScFv2-FN7-ScFv1, lane 3: ScFv3-FN10-ScFv1, lane 4: ScFv2-FN10-ScFv1, lane 5: Fab2H-FN7-ScFv1, lane 6: Fab2L-FN7-ScFv1, lane 7: Fab1L-FN10-Scfv2, lane 8: Fab1L-FN7-ScFv2, lane 9: ScFv3-FN7-Fc, lane 10: ScFv3-FN7-Fc-ScFv1, lane 11: Fab1H-FN10-Scfv3, and lane 12: Fab1L-FN7-Scfv3. FIG. 4C and FIG. 4D are SDS-PAGE of different components of GST-mFN7-His and GST-mFN7 purified by GSH column, respectively; and FIG. 4E is SDS-PAGE of different components of mFN7-4g-RSV-His and mFN7-5g-RSV-His purified by Ni column; where FT represents the flow-through fluid, W represents the liquid collected by cleaning the GSH column or Ni column, and E represents the eluent.

FIGS. 5A-C indicate the effects of different GCSF fusion proteins on NFS-60 cell proliferation.

FIG. 6 indicates the effects of hGH fusion protein on NB2-11 cell proliferation.

FIGS. 7A-C are ELISA results of RSV F protein in different fusion forms binding to motavizumab.

FIG. 8 shows killing activities of anti-HER2/anti-CD3 bispecific fusion protein on different cell lines by LDH release assay.

FIG. 9 shows the killing activity of anti-CD19/anti-CD3 bispecific fusion protein ScFv3-FN7-ScFv1 on different cell lines as measured by LDH release assay.

FIG. 10 shows the killing activity of anti-CD19/anti-CD3 bispecific fusion protein on NALM-6 cell line by FACS.

FIG. 11 is a PK plot of FN7-3g-GCSF-Fc concentrations in mouse plasma over time, where I.V means intravenous injection administration and S.C. means subcutaneous injection administration.

FIGS. 12A-B are PK plots of anti-HER2/anti-CD3 bispecific fusion protein concentrations in mouse plasma over time.

FIG. 13 shows the trend of neutrophils concentration in blood over time after injection of FN7-3g-GCSF-Fc (human GCSF as a positive control) into mice.

FIG. 14 is a plot of tumor mass (FIGS. 14A-B) and body weight (FIGS. 14C-D) over time after injection of ScFv2-FN7-ScFv1 and Fab2L-FN7-Scfv1 into tumor-bearing mice.

FIG. 15 is a plot of tumor diameter over time after injection of anti-CD19/anti-CD3 bispecific fusion protein ScFv3-FN7-ScFv1 into tumor-bearing mice.

FIG. 16 is a plot of concentrations of GH fusion protein FN7-3g-hGH-Fc in rat plasma by different administration modes, where I.V means intravenous injection administration and S.C. means subcutaneous injection administration.

FIG. 17 shows the trend of body weight over time in hypophysectomized male SD rats following subcutaneous injection of various doses of GH fusion protein (FN7-3g-hGH-Fc).

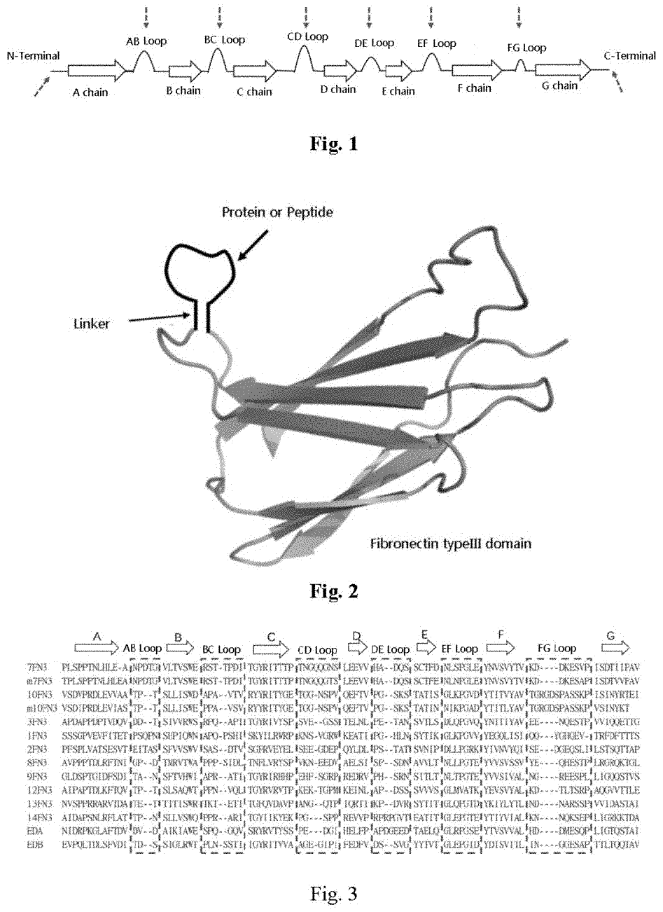

### DETAILED DESCRIPTION OF THE EMBODIMENTS

Fibronectin Type III Domain

Fibronectin type III domain refers to the type III domain of fibronectin. Fibronectin consists of 12 fibronectin type I domains, 2 type II domains and 15-17 type III domains. The sequence similarity of the different type III domains is not high (30% or less), but their secondary structures are highly conserved. The structure of fibronectin type III domain is similar with that of immunoglobulin. The sequence from N-terminal to C-terminal of protein includes: β or β-like chain A; loop AB; β or β-like chain B; loop BC; β or β-like chain C; loop CD; β or β-like chain D; loop DE; β or β-like chain E; loop EF; β or β-like chain F; loop FG; β or β-like chain G. The seven antiparallel β chains are arranged in two β lamellae, which form a stable core, and forming two “faces” consisting of loops connecting each β or β-like chain. As used in this application, “FN7” or “7FN3” refers to the fibronectin 7^{th} type III domain. “FN7” of the present application is the 7^{th} type III domain of mammalian fibronectin, preferably primate and rodent FN7, more preferably human FN7 and mouse FN7. The human FN7 preferably has the nucleic acid sequence shown in SEQ ID NO: 1 or the amino acid sequence shown in SEQ ID NO: 2, and the mouse FN7 preferably has the nucleic acid sequence shown in SEQ ID NO: 69 or the amino acid sequence shown in SEQ ID NO: 70. “FN10” or “10FN3” refers to the fibronectin 10^{th} type III domain. “FN10” of the present application is the 10^{th} type III domain of mammalian fibronectin, preferably primate and rodent FN10, more preferably human FN10 and mouse FN10. The human FN10 preferably has the nucleic acid sequence shown in SEQ ID NO: 3 or the amino acid sequence shown in SEQ ID NO: 4, and the mouse FN10 preferably has the nucleic acid sequence shown in SEQ ID NO: 71 or the amino acid sequence shown in SEQ ID NO: 72. Since the secondary structures of the different fibronectin type III domains are highly conserved (see FIG. 3), the skeleton proteins of the present application for use in physiologically active peptide fusion are not limited to FN7 (7FN3) and FN10 (10FN3), other fibronectin type III domains, such as AB loop, BC loop, CD loop, DE loop, EF loop, and/or FG loop of 3FN3, 1FN3, 2FN3, 8FN3, 9FN3, 12FN3, 13FN3, 14FN3, EDB or EDA may also be inserted with the physiologically active peptide, in addition that the N-terminal and/or C-terminal of these FN3s may also be fused to the physiologically active peptide by a linker.

Fibronectin type III domain suitable for use in the fusion proteins of the present application may be a wild-type fibronectin type III domain, or may be a native or artificial variant of a fibronectin type III domain, provided that the variant retains the same secondary structure with that of wild-type fibronectin type III domain. Thus, for example, human FN7 suitable for use in the fusion proteins of the present application may comprise an amino acid sequence having at least 80%, 85%, 90%, 91%, 92%, 93%, 94%, 95%, 96%, 97%, 98%, 99%, or 99.5% identity with the amino acid sequence as shown in SEQ ID NO: 2; human FN10 suitable for use in the fusion proteins of the present application may comprise an amino acid sequence having at least 80%, 85%, 90%, 91%, 92%, 93%, 94%, 95%, 96%, 97%, 98%, 99%, or 99.5% identity with the amino acid sequence as shown in SEQ ID NO: 4; and mouse FN7 suitable for use in the fusion proteins of the present application may comprise an amino acid sequence having at least 80%, 85%, 90%, 91%, 92%, 93%, 94%, 95%, 96%, 97%, 98%, 99%, or 99.5% identity with the amino acid sequence as shown in SEQ ID NO: 70. Mouse FN10 suitable for use in the fusion proteins of the present application may comprise an amino acid sequence having at least 80%, 85%, 90%, 91%, 92%, 93%, 94%, 95%, 96%, 97%, 98%, 99%, or 99.5% identity with the amino acid sequence as shown in SEQ ID NO: 72.

Physiologically Active Peptide

As used herein, “physiologically active peptide” is a general term for a polypeptide having a physiological effect in vivo, and a physiologically active polypeptide has common characteristics of the polypeptide structure and has various physiological activities. Physiologically active peptides may also include pharmacologically active polypeptides, such as polypeptides that correct abnormal pathological conditions caused by lack or excessive secretion of substances, which participate in the regulation of functions in vivo by regulating gene expression and physiological functions, and may also include general protein therapeutics. Vaccine antigen proteins capable of eliciting an immune system response upon administration to a human or animal (e.g., producing antibodies, or exhibiting activation of certain cells, particularly antigen presenting cells such as dendritic cells, T lymphocytes, B lymphocytes), or which can be bound by a particular antibody, are also within the scope of the “physiologically active peptides” herein. Therefore, in some aspects, “physiologically active peptides” are immunogenic polypeptides. Furthermore, the term “physiologically active peptide” is a concept that encompasses not only native polypeptides but also all derivatives thereof. Derivatives of physiologically active polypeptides may refer to those whose binding affinity for the natural receptor has been altered or whose physicochemical properties have been modified, such as increased water solubility and reduced immunogenicity, through chemical modifications such as amino acid substitutions, insertions and deletions, addition of glycans, removal of glycans, insertion of unnatural amino acids, insertion of rings and methyl residues. Derivatives of physiologically active polypeptides may also include artificial peptides engineered to have binding affinities for at least two different receptors (Chinese patent CN 103180338 B).

The “physiologically active peptide” of the present application may be a hormone, a cytokine, a vaccine antigen, an interleukin, an interleukin-binding protein, an enzyme, an antibody, a growth factor, a transcription factor, a blood factor, a structural protein, a ligand protein or receptor, a receptor antagonist, a cell surface antigen, an antibody or a antigen-binding fragment thereof, a toxic protein, human growth factor, a growth hormone releasing peptide, an interferon, an interferon receptor, a colony stimulating factor, a virus-derived vaccine antigen, a monoclonal antibody, a polyclonal antibody and an antibody fragment, glucagon-like peptides, a G protein-coupled receptor, interleukin, an interleukin receptor, an enzyme, an interleukin binding protein, a cytokine binding protein, a macrophage activating factor, a B cell factor, a T cell factor, protein A, an allergy inhibitor, a cell necrosis glycoprotein, an immunotoxin, a lymphotoxin, a tumor necrosis factor, a tumor suppressor, a metastasis growth factor, α-1 antitrypsin, albumin, α-lactalbumin, apolipoprotein-E, erythropoietin, a highly glycosylated erythropoietin, an angiopoietin, hemoglobin, thrombin, a thrombin receptor activating peptide, thrombomodulin, factor VII, factor VIIa, factor VIII, factor IX, factor XIII, a plasminogen activator, a fibrin-binding peptide, urokinase, streptokinase, hirudin, protein C, C-reactive protein, a renin inhibitor, a collagenase inhibitor, a superoxide dismutase, leptin, a platelet-derived growth factor, an epithelial growth factor, an epidermal growth factor, an angiostatin, an angiotensin, a bone growth factor, a bone stimulating protein, calcitonin, insulin, atrial peptide hormone, cartilage-inducing factor, elcatonin, a connective tissue activating factor, a tissue factor pathway inhibitor, follicle-stimulating hormone, luteinizing hormone, luteinizing hormone releasing hormone, a nerve growth factor, parathyroid hormone, relaxin, secretin, a stomatomedin, an insulin-like growth factor, an adreno cortical hormone, glucagon, cholecystokinin, pancreatic polypeptide, gastrin releasing peptide, a corticotropin releasing factor, thyroid stimulating hormone, an autocrine motility factor, lactoferrin, tubocurarine, a receptor, a receptor antagonist or a cell surface antigen and the like.

In a specific embodiment, the physiologically active peptide is a peptide having a therapeutic or prophylactic effect.

In the fusion proteins of the present application, the fibronectin type III domain and the physiologically active peptide may be derived from the same species, but may also be derived from different species. Those skilled in the art can appropriately select and combine fibronectin type III domains and physiologically active peptides based on the use of the fusion protein. For example, when the fibronectin type III domain is used as a vaccine carrier to be fused with a physiologically active peptide as an immunogen, it is preferable to use a fibronectin type III domain derived from the same species as the object to which the vaccine is intended to be administered.

In addition, in the context of the present disclosure, “peptide”, “polypeptide”, and “protein” can be used interchangeably, unless those skilled in the art judge otherwise based on the context. Those skilled in the art will recognize that “physiologically active peptide” does not limit the specific length or spatial structure of the peptide.

In one aspect, the fusion protein of the present application includes a first physiologically active peptide and a second physiologically active peptide. The two may be the same peptide or different.

In an embodiment, the first physiologically active peptide is inserted within the fibronectin type III domain by a linker and the second physiologically active peptide is connected at the N-terminal or the C-terminal of the fibronectin type III domain by a linker.

In another embodiment, both the first physiologically active peptide and the second physiologically active peptide are inserted within the fibronectin type III domain via linkers. In a more specific embodiment, the first physiologically active peptide and the second physiologically active peptide are individually inserted in different positions of the fibronectin type III domain. In a further more specific embodiment, the first physiologically active peptide and the second physiologically active peptide are individually inserted in different loops of the fibronectin type III domain.

In a yet another embodiment, the first physiologically active peptide is fused to the N-terminal of the fibronectin type III domain by a linker and the second physiologically active peptide is fused to the C-terminal of the fibronectin type III domain by a linker.

In yet another embodiment, the first physiologically active peptide is fused to the N-terminal of the fibronectin type III domain by a linker and the second physiologically active peptide is fused to the N-terminal of the first physiologically active peptide by a linker.

In yet another embodiment, the first physiologically active peptide is fused to the C-terminal of the fibronectin type III domain by a linker and the second physiologically active peptide is fused to the C-terminal of the first physiologically active peptide by a linker.

Linker

As used herein, the term “linker” refers to a peptide that acts as a linker, but is not physiologically active, in contrast to “physiologically active peptides”. The “linker” of the present application has wide applicability and transferability. The “linker” includes rigid peptides, flexible peptides, and peptides between fully rigid and fully flexible. In the context of the present disclosure, a “rigid peptide” refers to a peptide consisting essentially of a non-loop secondary structure, such as an alpha helix and a beta sheet. A “flexible peptide” refers to a peptide that has no secondary structure or consists essentially of a loop secondary structure. By adjusting the proportion and/or arrangement of rigid units (structural units with non-loop secondary structures) and flexible units (structural units without secondary structures or with loop secondary structures), the rigidity of the linker can be finely regulated to form a peptide located between the complete rigidity and the complete flexibility so as to meet different requirements on the rigidity of the linker in the construction of the fusion protein. In the present application, the flexible peptide may be selected from, but not limited to: (SG4)ₙ, G₄(SG₄)ₙ or (G4S)ₙ, wherein n is a number not less than 1, preferably n=1, 2, 3, 4, 5, 7, 8, 9, 10, 15 or 20; (Gly)₈, (Gly)₆, GGGSGGGGS, GGGGSGGGS, GSAGSAAGSGEF, KESGSVSSEQLAQFRSLD, EGKSSGSGSESKST and the like. The rigid peptide is selected from, but not limited to: (EAAAK)ₙ, GGSG(AKLAALK)ₙ, (AKLAALK)ₙ or A(EAAAK)ₙA, wherein n is a number not less than 1, preferably n is selected from an integer from 1 to 10; QESLYVDLFDKF, ELARLIRLYFAL, AAQIRDQLHQLRELF, LQQKIHELEGLIAQH, LQDAKVLLEAAL, LSDLHRQVSRLV, LAKILEDEEKHIEWL, LKLELQLIKQYREAL, QLEKKLQALEKKLAQLEKKNQALEKKLAQ, ALKKELQANKKELAQLKKELQALKKELAQ, LAAVESELSAVESELASVESELAAC, CAALKSKVSALKSKVASLKSKVAAL, QLEKKLQALEKKLAQLEKKNQALEKKLAQ, LAAVESELSAVESELASVESELAAC, ELAALEAELAALEAGGSG, ELAALEAELAALEA, (ELAALEA)ₙGGSG, (ELAALEA)ₙ, ALKKELQANKKELAQLKKELQALKKELAQ, CAALKSKVSALKSKVASLKSKVAAL, GGSGAKLAALKAKLAALK, AKLAALKAKLAALK and the like.

Fab

“Fab” as used herein refers to a protein consisting of VH and CH1 domains of a heavy chain and VL and CL domains of a light chain of an immunoglobulin. Wherein the chain consisting of VH and CH1 domains is “Fab heavy chain” or “Fab-HC”, and the chain consisting of VL and CL domains is “Fab light chain” or “Fab-LC”.

Fusion or Linkage

“Fusion” or “linkage” means that the members (e.g., the physiologically active peptide and the fibronectin type III domain) are linked by peptide bonds, either directly or via one or more linkers.

### EXAMPLES

It should be noted that, in the case of no conflict, the embodiments in this application are merely examples, and are not intended to limit the present application in any manner. Unless otherwise specified, the experimental procedures used in the examples are those routinely used in the art, for example, see M. R. Green, J. Sambrook Molecular Cloning A LABORATORY MANUAL, 4^{th} edition, Cold Spring Harbor Laboratory Press, 2012.

### 1 Construction of Fusion Protein Expression Vector

### 1.1 Construction of Human Fibronectin Type III Domain Fusion Protein Expression Vector

PCR amplification was performed on human GCSF (granulocyte colony stimulating factor) (SEQ ID NO: 5), human GH (growth hormone) (SEQ ID NO: 7), anti-CD3 Fab-HC (SEQ ID NO: 31), anti-CD3 Fab-LC (SEQ ID NO: 33), anti-HER2 Fab-HC (SEQ ID NO: 35), anti-HER2 Fab-LC (SEQ ID NO: 37), anti-CD3 scFv (SEQ ID NO: 63), anti-HER2 scFv (SEQ ID NO: 65), anti-CD19 scFv (SEQ ID NO: 67), and RSV F (SEQ ID NO: 115) genes (all synthesized by IDT). Using standard molecular biology techniques, both terminus of human GCSF, human GH gene or RSV F gene were cloned into different loops of a human fibronectin (fibronectin, FN) 10^{th} type III domain (FN10) or 7^{th} type III domain (FN7) through linkers (6 loops from the N-terminal to the C-terminal of the fibronectin type III domain are AB loop, BC loop, CD loop, DE loop, EF loop, and FG loop in turn) to give FN10-6g-CCSF, FN7-3g-GCSF, FN7-3g-hGH, FN7-3a-GCSF, FN7-3a-hGH, FN10-3g-GCSF, FN10-3a-GCSF, FN7-6g-GCSF, FN10-6a-GCSF, FN10-1g-RSV, FN10-2g-RSV, FN10-3g-RSV, FN10-4g-RSV, FN10-5g-RSV, FN10-6g-RSV fragments (wherein the number “1” in “1a” indicates that the insertion position is within the first loop, i.e. the AB loop starting from the N-terminal of the fibronectin type III domain, and “a” indicates that the linker used is an α-helix linker; the number “6” in “6g” indicates that the insertion position is within the 6th loop, i.e. the FG loop of the fibronectin type III domain starting from the N-terminal, and “g” indicates that the linker used is a GS linker, and so on, which is applicable to all examples). The two gene fragments FN7-3g-GCSF and FN7-3g-hGH described above were fused to the N-terminal of human IgG1 Fc (containing substitutions E233P, L234V, L235A, ΔG236, A327G, A330S and P331S) by linkers to give FN7-3g-GCSF-Fc and FN7-3g-GH-Fc, respectively. Overlapping PCR was performed to clone the anti-CD3 Fab-HC, anti-CD3 Fab-LC, anti-HER2 Fab-HC, anti-HER2 Fab-LC, anti-CD3 scFv1, anti-HER2 scFv, and anti-CD19 scFv genes to the N-and/or C-terminal of FN7 or FN10 via linkers. The resulting fusion protein gene construct fragment was cloned into a pFuse vector (InvivoGen, CA) using in-frame ligation to construct a fusion protein expression vector, which were sequenced for verification. The nucleic acid and amino acid sequences of each fusion protein construct constructed therefrom are as shown in SEQ ID NO: 9-SEQ ID NO: 62, and SEQ ID NO: 107-SEQ ID NO: 114, see Table 1.

### 1.2 Construction of Mouse Fibronectin Type III Domain Fusion Protein Expression Vectors

RSV F, mouse mFN7 (SEQ ID NO: 69), mouse mFN10 (SEQ ID NO: 71) genes (all synthesized by IDT) were PCR amplified. Using a standard molecular biology method, RSV genes were inserted into different loops of mouse fibronectin 10^{th} type III domain (mFN10) or 7^{th} type III domain (mFN7) at both terminus via linkers, to give mFN7-1g-RSV, mFN7-2g-RSV, mFN7-3g-RSV, mFN7-4g-RSV, mFN7-5g-RSV, mFN7-6g-RSV, mFN10-1g-RSV, mFN10-2g-RSV, mFN10-3g-RSV, mFN10-4g-RSV, mFN10-5g-RSV, and mFN10-6g-RSV fusion protein gene construct fragments. The fusion protein gene construct fragments were cloned into pGEX6p-1 (GE healthcare), pET28a (Novagen) or pFuse vector (InvivoGen), to construct a fusion protein expression vector, which were sequenced for verification. The nucleic acid and amino acid sequences of each fusion protein constructed therefrom are shown in SEQ ID NO: 77-SEQ ID NO: 80, SEQ ID NO: 83-SEQ ID NO: 94, SEQ ID NO: 97-SEQ ID NO: 100, and SEQ ID NO: 103-SEQ ID NO: 106.

TABLE 1
The structural modules used to construct the fusion protein
in Example 1 and the sequence of each constructed fusion
protein constructs are shown in the following table.

| | | Nucleic acid | Amino acid |
|---|---|---|---|
| | | sequence | sequence |
| | | SEQ ID NO: | SEQ ID NO: |
| Construction Module | | | |
| FN7 | | 1 | 2 |
| FN10 | | 3 | 4 |
| hGCSF | | 5 | 6 |
| hGH | | 7 | 8 |
| mFN7 | | 69 | 70 |
| mFN10 | | 71 | 72 |
| RSV F | | 115 | 116 |
| Scfv1 (anti-CD3 scfv) | | 63 | 64 |
| Scfv2 (anti-HER2 scfv) | | 65 | 66 |
| Scfv3 (anti-CD19 scfv) | | 67 | 68 |
| Fab1 | Fab1-HC | 31 | 32 |
| (anti-CD3 Fab) | Fab1-LC | 33 | 34 |
| Fab2 | Fab2-HC | 35 | 36 |
| (anti-HER2 Fab) | Fab2-LC | 37 | 38 |
| Construct | | | |
| FN10-6g-GCSF | | 9 | 10 |
| FN7-3g-GCSF | | 11 | 12 |
| FN7-3g-GCSF-Fc | | 13 | 14 |
| FN7-3g-hGH | | 15 | 16 |
| FN7-3g-hGH-Fc | | 17 | 18 |
| FN7-3a-GCSF | | 19 | 20 |
| FN7-3a-hGH | | 21 | 22 |
| FN10-3g-GCSF | | 23 | 24 |
| FN10-3a-GCSF | | 25 | 26 |
| FN7-6g-GCSF | | 27 | 28 |
| FN10-6a-GCSF | | 29 | 30 |
| ScFv2-FN7-ScFv1 | | 39 | 40 |
| ScFv2-FN10-ScFv1 | | 41 | 42 |
| ScFv3-FN7-ScFv1 | | 43 | 44 |
| ScFv3-FN10-ScFv1 | | 45 | 46 |
| ScFv3-FN7-Fc | | 47 | 48 |
| ScFv3-FN7-Fc-ScFv1 | | 49 | 50 |
| Fab2H-FN7-Scfv1 | Chain 1 (Fab2-HC-FN7-ScFv1) | 51 | 52 |
| | Chain 2 (Fab2-LC) | 37 | 38 |
| Fab2L-FN7-Scfv1 | Chain 1 (Fab2-HC) | 35 | 36 |
| | Chain 2 (Fab2-LC-FN7-ScFv1) | 53 | 54 |
| Fab1L-FN7-Scfv2 | Chain 1 (Fab1-HC) | 31 | 32 |
| | Chain 2 (Fab1-LC-FN7-ScFv2) | 55 | 56 |
| Fab1L-FN10-Scfv2 | Chain 1 (Fab1-HC) | 31 | 32 |
| | Chain 2 (Fab1-LC-FN10-Scfv2) | 57 | 58 |
| Fab1L-FN7-Scfv3 | Chain 1 (Fab1-HC) | 31 | 32 |
| | Chain 2 (Fab1-LC-FN7-Scfv3) | 59 | 60 |
| Fab1H-FN10-Scfv3 | Chain 1 (Fab1-HC-FN10-Scfv3) | 61 | 62 |
| | Chain 2 (Fab1-LC) | 33 | 34 |

| | | |
|---|---|---|
| GST-mFN7-His | 73 | 74 |
| GST-mFN7 | 75 | 76 |
| GST-mFN7-RSV-1 | 77 | 78 |
| GST-mFN7-RSV-2 | 79 | 80 |
| mFN7-His | 81 | 82 |
| mFN7-RSV-1-His | 83 | 84 |
| mFN7-RSV-2-His | 85 | 86 |
| mFN7-RSV-3-His | 87 | 88 |
| mFN7-RSV-4-His | 89 | 90 |
| mFN7-RSV-5-His | 91 | 92 |
| mFN7-RSV-6-His | 93 | 94 |
| mFN10-His | 95 | 96 |
| mFN10-RSV-6-His | 97 | 98 |
| His-mFN10-RSV-6 | 99 | 100 |
| His-mFN10 | 101 | 102 |
| mFN10-OVA-3-RSV-6-His | 103 | 104 |
| His-mFN10-OVA-3 | 105 | 106 |
| FN10-1g-RSV | 107 | 108 |
| FN10-2g-RSV | 109 | 110 |
| FN10-3g-RSV | 111 | 112 |
| FN10-6g-RSV | 113 | 114 |

### 2. Expression and Purification of Fusion Proteins

### 2.1 Expression and Purification in Mammalian Cells

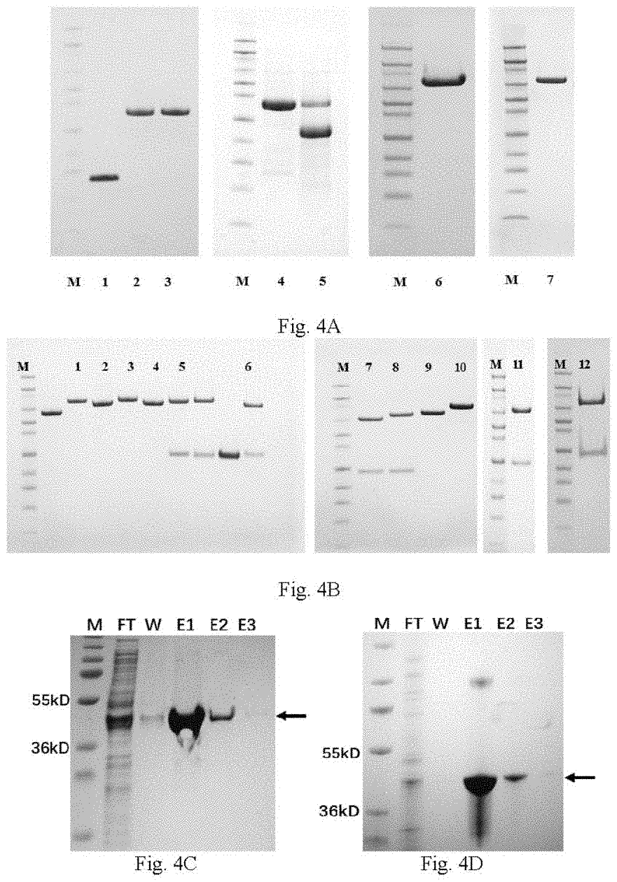

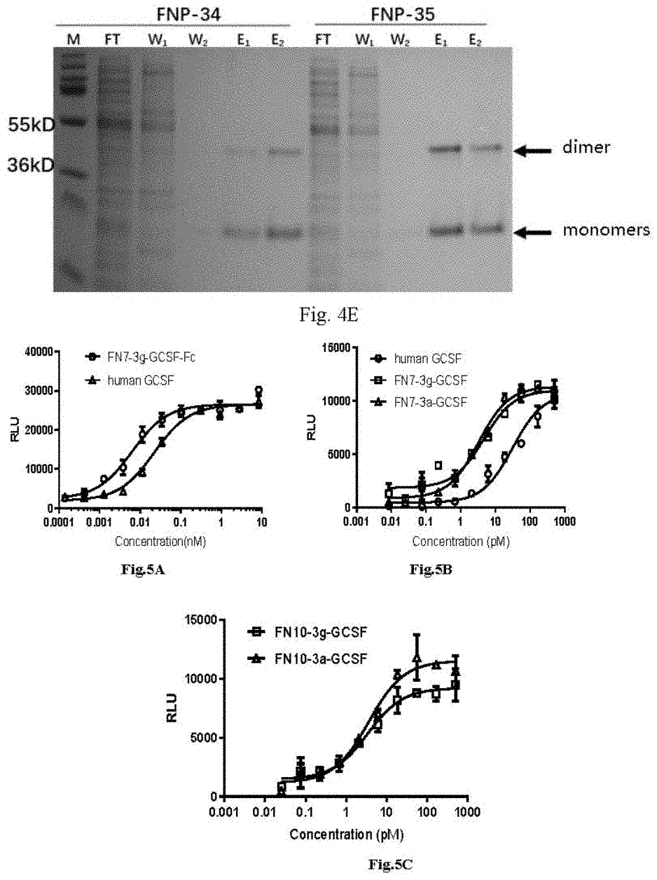

The fusion protein eukaryotic expression vectors constructed in Examples 1.1 and 1.2 were transiently transfected into FreeStyle HEK293 cells: 28 ml FreeStyle HEK 293 (3×10⁷ cells/ml) was inoculated into a 125 ml cell culture flask; the plasmid was diluted with 1 ml Opti-MEM, added to 1 ml Opti-MEM containing 60 μl 293 fectin (Invitrogen, Inc), and left at room temperature for 30 min; the plasmid-293 fectin mixture was added to the cell culture medium, and cultured at 37° C., 5% CO₂ under 125 rpm. Cell culture supernatant was collected 48 h and 96 h after transfection, purified by Protein A/G (Thermo Fisher Scientific, IL) or Ni-NTA, and detected by SDS-PAGE. The results are as shown in FIGS. 4A and 4B.

### 2.2 Expression and Purification in Prokaryotic Cells

The prokaryotic expression vector constructed in Example 1.2 was transformed into BL21 competence. The bacterial solution was expanded (37° C., 200 rpm); when cultured to OD 0.7, 0.1 mM IPTG was added to induce for expression at 30° C. for 6 h. The thalli were collected and thawed, and disrupted under high pressure, the suspension was collected, and centrifuged at 12000 rpm for 30 min.

For fusion proteins cloned into pGEX6p-1 vector, the supernatant was passed through a GST gravity column twice. The column was washed with equilibration buffer for 50 column volumes (20 mM Tris 500 mM NaCl pH 7.5), and eluted with 10 mM GSH for 10 column volumes firstly; and then the remaining proteins were eluted with 20 mM GSH. Samples of each fraction were collected and run on SDS gel for detection.

For fusion proteins cloned into pET28a vector, the supernatant was passed through a Ni column twice. The column was washed with equilibration buffer containing 40 mM imidazole for 50 column volumes (20 mM Tris 500 mM NaCl pH7.5), and eluted with 200M imidazole for 10 column volumes firstly; and then the remaining proteins were eluted with 500 mM GSH. Samples of each fraction were collected and run on SDS gel for detection. The results are as shown in FIGS. 4C, 4D and 4E.

### 3. In Vitro Activity Studies

### 3.1 Effect of GCSF Fusion Protein on Proliferation of NFS-60 Cells

The fusion proteins FN10-3g-GCSF, FN10-3a-GCSF, FN7-3a-GCSF, FN7-3g-GCSF, FN7-3g-GCSF-Fc prepared in Example 2 were taken for study of effects on the proliferation activity of NFS-60 cells.

The specific steps include culture of NFS-60 (ATCC, USA) cells (RPMI-1640 medium: 10% fetal bovine serum, 0.05 mM 2-mercaptoethanol, 62 ng/ml hGM-CSF). NFS-60 cells were washed three times with RPMI-1640 incomplete medium and the cell density was adjusted to 1.5×10⁵ cells/ml with RPMI-1640 complete medium (containing 10% fetal bovine serum, 0.05 mM 2-mercaptoethanol) and added to 96-well plates (100 μl/well) before the proliferation activity assay was started. Gradient diluted human GCSF, FN10-3g-GCSF, FN10-3a-GCSF, FN7-3a-GCSF, FN7-3g-GCSF, or FN7-3g-GCSF-Fc protein were added to 96-well plates and cultured at 37° C., 5% CO₂. After 72 h, 1/10 volume of AlamarBlue (Invitrogen) was added and incubated at 37° C. for 4 h, and the fluorescence value at 595 nm was measured.

The results are as shown in FIG. 5. Fusion proteins FN7-3g-GCSF, FN7-3a-GCSF, FN7-3g-GCSF-Fc can promote the proliferation of NFS-60 cells significantly stronger than human GCSF (see FIGS. 5A and 5B), and there is no significant difference in proliferation ability between FN7-3g-GCSF and FN7-3a-GCSF, FN10-6g-GCSF and FN10-6a-GCSF.

### 3.2 Effect of hGH Fusion Protein on Proliferation of NB2-11 Cells

Rat NB2-11 cells (Sigma) were cultured (RPMI medium: containing 10% horse serum (Life Technologies, CA), 55 μM 2-mercaptoethanol (Life Technologies, CA)); the cell density was adjusted to 2.5×10⁵ cells/ml, and plated on a 96-well plate (200 μl/well). Gradient diluted hGH or FN7-3g-hGH-Fc protein were added to 96-well plates and cultured at 37° C., 5% CO₂. After 72 h, 20 μl of Prestoblue was added to each well, and the fluorescence value at 590 nm (excitation wavelength 550 nm) was read.

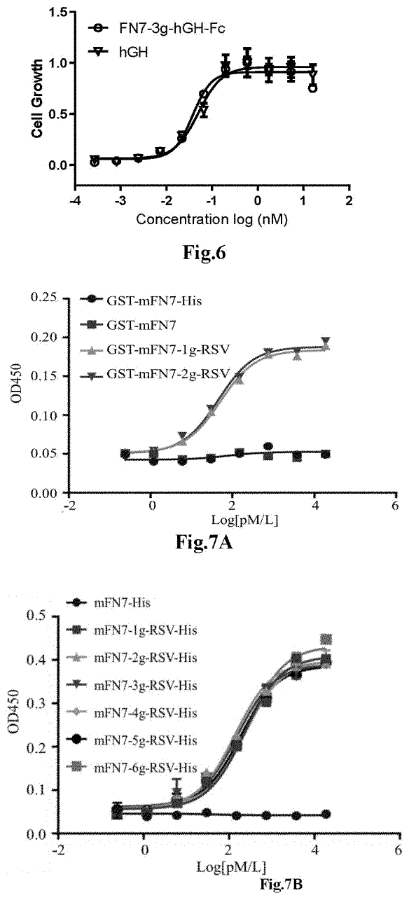

The results are as shown in FIG. 6. FN7-3g-hGH-Fc is similar to hGH in promoting the proliferation of NB2-11 cells.

### 3.3 ELISA of RSV F Fusion Protein Binding MOTA

The motavizumab (neutralizing antibody to RSV F protein, expressed in this laboratory) (DPBS buffer, pH7.4) was coated on the 96-well plate, and incubated at 4° C. overnight; followed by blocking with DPBST containing 2% skim milk powder for 1 hour at room temperature, the plate was washed with DPBS containing 0.05% Tween-20 three times, then added with gradient diluted RSV F fusion protein (RSV F protein (purchased from Sino Biological) as a positive control) and incubated at room temperature for 2 hours; the plate was washed with DPBS containing 0.05% Tween-20 4-5 times; HRP conjugated anti-His (Genscript) secondary antibody was added and incubated for 2 hours at room temperature. After washing with DPBS containing 0.05% Tween-20 4-5 times, TMB (BioLegend) was developed and read at OD450. Log (agonist) vs. response models of Prizm Graphpad software was used for nonlinear regression of data.

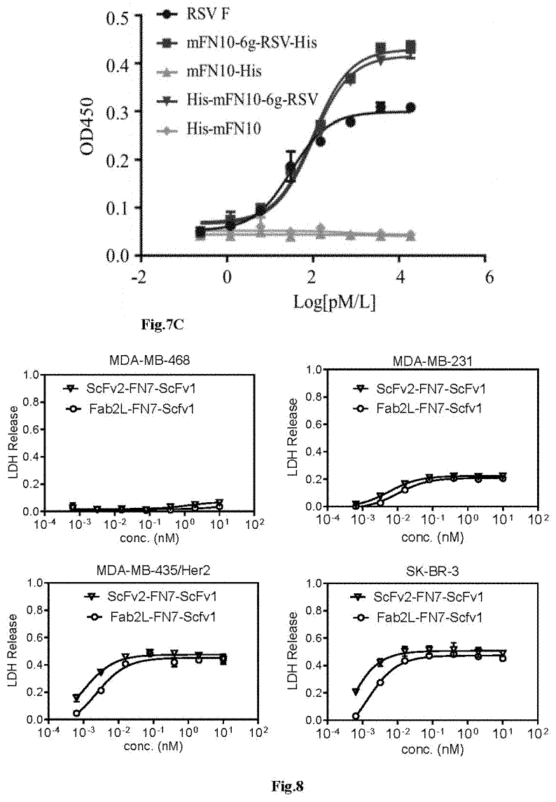

The results are as shown in FIG. 7. RSV F fusion proteins inserted into different positions of mouse fibronectin type III domain (GST-mFN7-1g-RSV, GST-mFN7-2g-RSV, mFN7-1g-RSV-His, mFN7-2g-RSV-His, mFN7-3g-RSV-His, mFN7-4g-RSV-His, mFN7-5g-RSV-His, mFN7-6g-RSV-His, mFN10-6g-RSV-His, His-mFN10-6g-RSV) have similar affinity to the antibody motavizumab and are all stronger than commercial recombinant RSV F protein, suggesting that the epitope peptide of RSV F protein fused into the loop inside fibronectin type III domain can still maintain its original conformation, and retain high affinity to motavizumab antibodies.

### 3.4 Detection of Cytotoxicity of Anti-HER2/Anti-CD3 Bispecific Antibody Fusion Protein

Peripheral blood was collected from healthy volunteers and peripheral blood mononuclear cells (PBMCs) were isolated by Ficoll-Hypaque (GE Healthcare) gradient centrifugation and resuspended in RPMI 1640/10% FBS complete medium. PBMCs were incubated with solid phase bound anti-CD3 (Clone OKT3, eBiosciences), 2 μg/mL anti-CD28 (Clone CD 28.2, eBiosciences) at 37° C., and after 48 hours activated T cell expansion was stimulated by the addition of 20 U/ml IL 2 (R&D Systems).

MDA-MB-468, MDA-MB-231, MDA-MB-435/HER2, SK-BR-3 cells were cultured in DMEM complete medium (containing 10% FBS, 1% Penicillin/Streptomycin). After trypsinization, they were incubated with the above activated T cells at a ratio of 10:1 (T cell density 10⁶ cells/mL, target cell density 10⁵ cells/mL), respectively. Followed by adding gradient diluted Fab2L-FN7-Scfv1 or ScFv2-FN7-ScFv1, they were incubated at 37° C., 5% CO₂ for 24 hours. The LDH content of each culture supernatant was measured by Cytotox-96 nonradioactive cytotoxicity assay kits (Promega). OD value at 490 nm was read by SpectraMax 250. Cytotoxicity (% expressed) was calculated as follows:

% cytotoxicity=(absorbance experimental−absorbance spontaneous average)/(absorbance maximum killing average−absorbance spontaneous average).

Wherein, the maximum killing is the LDH content in the supernatant of target only cells; spontaneous killing is the LDH content in the supernatant of cells containing target and effector cells (T cells) without fusion protein.

The results are as shown in FIG. 8. Fab2L-FN7-Scfv1 and ScFv2-FN7-ScFv1 have strong killing effects on HER2-positive target cells MDA-MB-231, MDA-MB-435/Her2 and SK-BR-3 cells, while for HER2-negative MDA-MB-468 cells have no effect.

### 3.5 Detection of Cytotoxicity of Anti-CD19/Anti-CD3 Bispecific Antibody Fusion Protein

### 3.5.1 LDH Release Assay

Peripheral blood was collected from healthy volunteers and peripheral blood mononuclear cells (PBMCs) were isolated by Ficoll-Hypaque (GE Healthcare) gradient centrifugation and resuspended in RPMI 1640/10% FBS complete medium. PBMCs were incubated with solid phase bound anti-CD3 (Clone OKT3, eBiosciences), 2 μg/mL anti-CD28 (Clone CD 28.2, eBiosciences) at 37° C., and after 48 hours activated T cell expansion was stimulated by the addition of 20 U/ml IL 2 (R&D Systems).

NALM-6 and HT-29 cells were cultured in DMEM complete medium (containing 10% FBS, 1% Penicillin/Streptomycin). After trypsinization, they were incubated with the above activated T cells at a ratio of 10:1 (T cell density 10⁶ cells/mL, target cell density 10⁵ cells/mL). Followed by adding gradient diluted ScFv3-FN7-ScFv1, they were incubated at 37° C., 5% CO₂ for 24 hours. The LDH content of each culture supernatant was measured by Cytotox-96 nonradioactive cytotoxicity assay kits (Promega). OD value at 490 nm was read by SpectraMax 250. Cytotoxicity (% expressed) was calculated as follows:

% cytotoxicity=(absorbance experimental−absorbance spontaneous average)/(absorbance maximum killing average−absorbance spontaneous average).

Wherein, the maximum killing is the LDH content in the supernatant of target only cells; spontaneous killing is the LDH content in the supernatant of cells containing target and effector cells (T cells) without fusion protein.

The results are as shown in FIG. 9. ScFv3-FN7-ScFv1 has a strong killing effect on CD19-positive Nalm-6 cells, but has little effect on CD19-negative HT-29 cells.

### 3.5.2 Fluorescent Staining

Peripheral blood was collected from healthy volunteers and peripheral blood mononuclear cells (PBMCs) were isolated by Ficoll-Hypaque (GE Healthcare) gradient centrifugation and resuspended in RPMI 1640/10% FBS complete medium. PBMCs were incubated with solid phase bound anti-CD3 (Clone OKT3, eBiosciences), 2 μg/mL anti-CD28 (Clone CD 28.2, eBiosciences) at 37° C., and after 48 hours activated T cell expansion was stimulated by the addition of 20 U/ml IL 2 (R&D Systems).

Nalm-6 cells were cultured in DMEM complete medium (containing 10% FBS, 1% Penicillin/Streptomycin). Nalm-6 cells were plated in 24-well plates and incubated with activated T cells at a ratio of 5:1 (T cell density 5*10⁵ cells/mL, target cell density 10⁵ cells/mL). Followed by adding gradient diluted ScFv3-FN7-ScFv1, ScFv3-FN10-ScFv1 or BiTE, the cells were incubated at 37° C., 5% CO₂ for 24 hours. After staining with CellTracer Orange CMRA Dye (Life Technology), fluorescence was observed under a FITC (for CSFE) filter and the number of viable Nalm-6 B cells was counted.

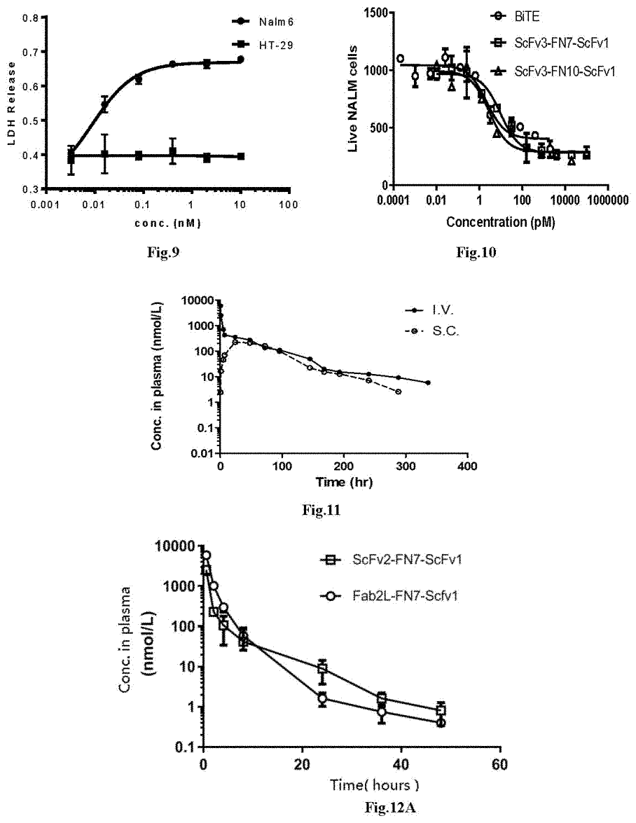

The results are as shown in FIG. 10. Both ScFv3-FN7-ScFv1 and ScFv3-FN10-ScFv1 can effectively inhibit the activity of Nalm-6 cells, and their effects are consistent with BiTE.

### 4. Pharmacokinetics in Mice

### 4.1 PK of GCSF Fusion Protein in Mice

CD1 mice (3 per group) were injected intravenously (I.V.) or subcutaneously (S.C.) with FN7-3g-GCSF-Fc (2 mg/kg). Blood was taken every day 0-14 days after injection, and ELISA was performed with anti-human IgG Fc antibody (KPL) and anti-hGCSF antibody (Abbiotec). ELISA readings of blood samples taken 30 min after injection were taken as the first time point.

The results are as shown in FIG. 11. Whether FN7-3g-GCSF-Fc is injected subcutaneously (S.C.) or intravenously (I.V.), its concentration in the body slowly decreases, indicating that it has a longer half-life.

### 4.2 PK of Anti-HER2/Anti-CD3 or Anti-CD19/Anti-CD3 Bispecific Fusion Protein in Mice

CD1 mice (6 per group) were injected intravenously with 0.2 mg anti-HER2/anti-CD3 bispecific fusion protein (ScFv2-FN7-ScFv1 and Fab2L-FN7-Scfv1) or 0.2 mg anti-CD19/anti-CD3 bispecific fusion protein (ScFv3-FN7-ScFv1) (dissolved in PBS, pH7.4). After 5 min, 15 min, 30 min, 1 h, 2 hrs, 4 hrs, 6 hrs, 8 hrs, 24 hrs, 32 hrs and 48 hrs respectively, 75 μl of heparin anticoagulant was collected from the eye socket and stored in dry ice. Anticoagulant blood was centrifuged at 12000 rpm for 3 min in a laboratory. The plasma was plated in 96-well plates, and the concentration of the fusion protein was detected in plasma by the solid-phase binding hErbB2-Fc (R&D systems) primary antibody and HRP anti-human Kappa (Abcam) secondary antibody. The elimination half-life was calculated by taking the last four time point data into the first order equation A=A₀ e^{-kt} (wherein, A₀ is the initial concentration, t is the time, and k is the first order rate constant).

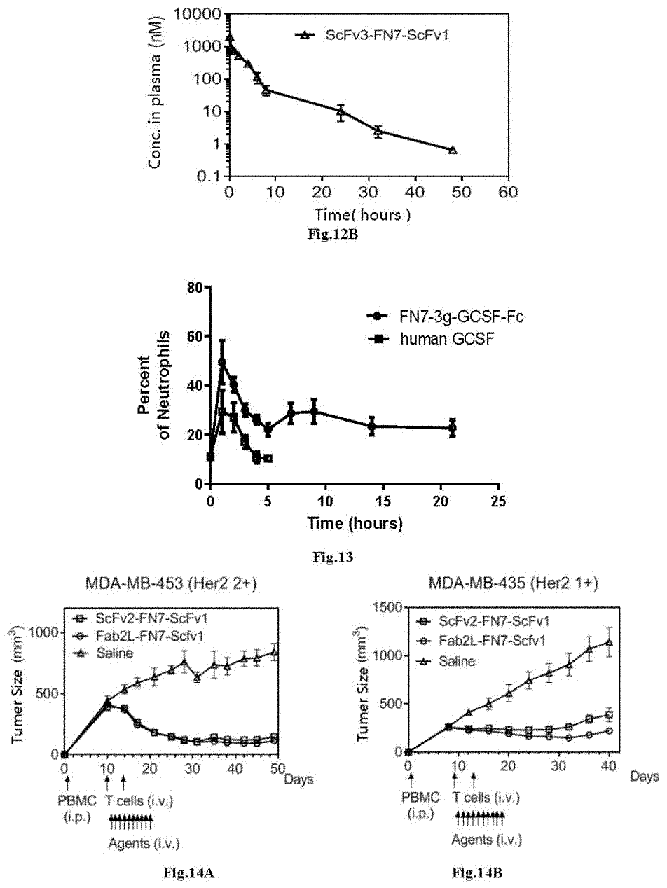

The results are as shown in FIG. 12a and FIG. 12b. The concentrations of ScFv2-FN7-ScFv1, Fab2L-FN7-Scfv1FNP-14 and ScFv3-FN7-ScFv1 in plasma decrease over time, with half-lives between 20-30 hours, much higher than that of Blinatumomab (BiTE) (1.5-2.1 hours).

### 5. Pharmacodynamics in Mice

### 5.1 Study on PD of GCSF Fusion Protein in Mice

BALB/c mice (3 per group) were injected subcutaneously with a single dose of human GCSF (10 μg/kg) or FN7-1g-GCSF-Fc (50 μg/kg). Blood samples were collected 0-21 days after injection. Neutrophil fractions were detected by FITC anti-CD 45 (Miltenyi Biotec), PE anti-CD 11b (Miltenyi Biotec) and APC anti-Ly-6G (BD Biosciences) antibody via flow cytometry.

The results are as shown in FIG. 13. Within 1 day after GCSF injection, neutrophils in blood increased rapidly, then decreased rapidly, and neutrophils could not be detected on the 6th day; however, the change trend of neutrophils in FN7-1g-GCSF-Fc injection group was consistent with that of GCSF, but from the 6th day to the 21th day, the fraction of neutrophils was almost the same as that of 6th day, suggesting that FNP-03 plays a role continuously.

### 5.2 Anti-HER2/Anti-CD3 Bispecific Fusion Protein

The ability of the fusion protein to inhibit tumor mass in tumor-bearing mice was examined in 6-8 week old female NOD-SCID-γ mice (NOD.Cg-prkdcscid II2rgtmlwjl/SzJ; Jackson Laboratory) and human breast cancer cells HER2 2+(MDA-MB-453), and HER2 1+(MDA-MB-435)].

HER2 2+ tumor model: 5×10⁶ MDA-MB-453 cells were resuspended in 50% Matrigel (BD Bioscience) and injected subcutaneously into the right flank of mice. The next day after injection, 2×10⁷ freshly prepared PBMCs were injected intraperitoneally; meanwhile, PBMCs were stimulated in vitro with solid phase bound anti-CD3 antibody (clone OKT3, eBioscience), 2 μg/mL anti-CD28 antibody (clone CD28.2, eBioscience), and 50 IU/mL recombinant human IL-2 (R&D Systems). 9 days and 12 days after tumor cell inoculation, 2×10⁷ activated T cells in vitro were injected intraperitoneally; when the tumor mass size reached 200-300 mm³, ScFv2-FN7-scFv1 or Fab2L-FN7-Scfv1 (1 mg/kg) or saline were injected intravenously daily for 10 days. Mouse body weights were measured daily.

HER2 1+ tumor model: 5×10⁶ MDA-MB-435 cells were resuspended in 50% Matrigel (BD Bioscience) and injected subcutaneously into the right flank of mice. The next day after injection, 2×10⁷ freshly prepared PBMCs were injected intraperitoneally. 9 days and 12 days after tumor cell inoculation, 2×10⁷ activated T cells in vitro were injected intraperitoneally; when the tumor mass size reached 200-300 mm³, ScFv2-FN7-scFv1 or Fab2L-FN7-Scfv1 (1 mg/kg) (1 mg/kg) or saline were injected intravenously daily for 10 days. The body weights of the mice were measured every day, and the size of tumor masses in all experimental mice were measured with calipers, twice a week. Tumor volume is calculated as follows: tumor volume=width*length*height.

The results are as shown in FIG. 14. After treatment with ScFv2-FN7-ScFv1 or Fab2L-FN7-Scfv1 (1 mg/kg), the tumor masses of MDA-MB-453 (HER2 2+) tumor-bearing mice and MDA-MB-435 (HER2 1+) tumor-bearing mice decreased significantly, and their body weights did not change significantly, suggesting that these fusion proteins not only have better tumor suppressor activity but also have better safety.

### 5.3 Anti-CD19/Anti-CD3 Bispecific Fusion Protein

The ability of anti-CD19/anti-CD3 bispecific fusion proteins to inhibit tumor mass in tumor-bearing mice was examined in NSG mice inoculated with Nalm-6 cells.

NSG mice were inoculated with 5×10⁵ Nalm-6 cells (expressing GFP) (day 0). Six (6) days after inoculation, 4×10⁷ PBMC were infused intravenously, and 6 hours later ScFv3-FN7-ScFv1 was infused intravenously. The body weights of the mice were measured daily, and when the body weights of the mice were reduced by more than 15% before the experiment, the mice were sacrificed. The tumor burden was measured by IVIS and was expressed as the radius of the region of interest (ROI).

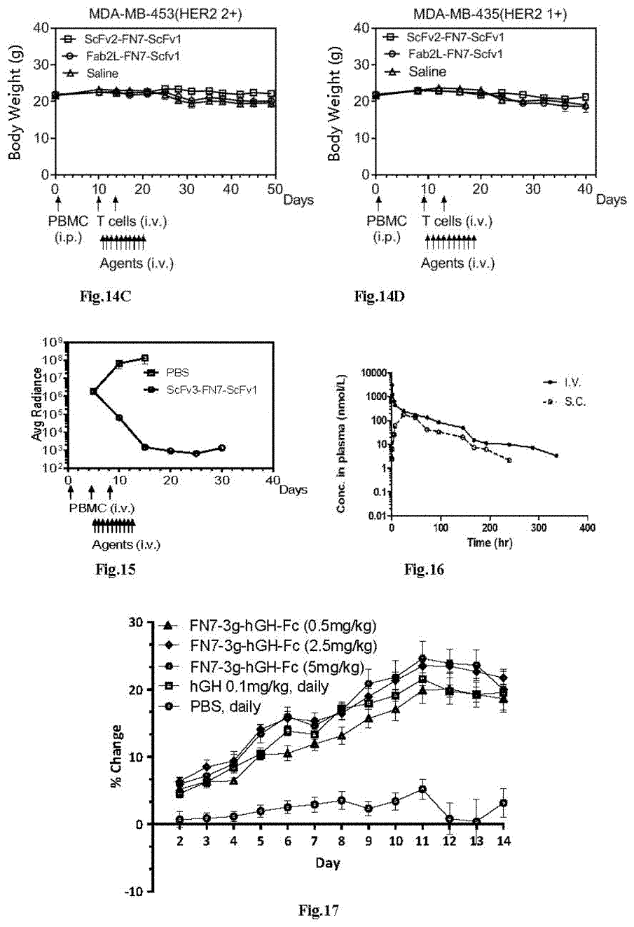

The results are as shown in FIG. 15. After infusion of ScFv3-FN7-ScFv1, the tumor radii of tumor-bearing NSG mice decreased significantly, while in PBS infusion group, the tumor continued to increase, suggesting that ScFv3-FN7-ScFv1 has a significant tumor-inhibiting effect.

### 6. Pharmacokinetics of GH Fusion Protein in Rats

FNP-05 was injected intravenously (I.V.) or subcutaneously (S.C.) into SD female rats (3 per group). Heparin anticoagulant blood was collected from the tail vein or saphenous vein, and the blood collection time was as follows: 30 min, 1 h, 2 hrs, 4 hrs, 6 hrs, 24 hrs, 48 hrs, 3 d, 4 d, 6 d, 8 d, 10 d, 12 d and 14 d. After centrifugation, the plasma was collected and stored at −80° C. for later use. Plasma hGH content is determined as follows (hGH human Direct ELISA kit; Life Technology) as follows: goat anti-human IgG Fc (Abcam, Mass.) was coated on maxisorb ELISA plates, incubated at 37° C. for 1 h, blocked with 5% BSA, and incubated with gradient diluted plasma for 1 h at room temperature. The unbound plasma was washed off. Primary antibodies biotinylated polyclonal anti-hGH antibodies (R&D systems, MN) were added and incubated for 1 h, and the plates washed 3 times. The streptavidin-HRP conjugate (Thermo Fisher Scientific, IL) was added and incubated for 1 h at room temperature, and the plates were washed three times. QuantaBIa fluorogenic ELISA substrate (Thermo Fisher Scientific, IL) was added and the fluorescence signal was detected by SpectraMax. The content of hGH in plasma was calculated from a standard curve (horizontal ordinates are hGH concentrations and vertical coordinates are fluorescence signal values). Pharmacokinetic parameters were estimated using a modeling program WinNonlin (Pharsight).

The results are as shown in FIG. 16. Whether FN7-3g-hGH-Fc is injected subcutaneously (S.C.) or intravenously (I.V.), it stays in the body for a longer time, reaching 10 days and 14 days, respectively, suggesting that it has a longer half-life.

### 7. Pharmacodynamics of GH Fusion Protein in Rats

The hypophysectomized male SD rats (8 rats in total) were injected with human GH (0.1 mg/ml, administered daily) and different concentrations of FN7-3g-hGH-Fc (0.5 mg/kg, 2.5 mg/kg, 5.0 mg/kg; twice a week) subcutaneously, and the body weights were measured daily.

The results are as shown in FIG. 17. The body weights of SD rats injected with PBS almost changed little, and the weight change of mice injected with different concentrations of FN7-3g-hGH-Fc twice a week was consistent with the change trend of the body weight of mice administrated with 0.1 mg/kg hGH daily, suggesting that FN7-3g-hGH-Fc can achieve similar effects to hGH.

### 8. Thermodynamic Stability Test

The fusion proteins were tested for thermodynamic stability using a fluorescence-based Protein Thermal Shift Assay (Applied Biosystems) according to the manufacturer's instructions. The sample (0.5 mg/ml, dissolved in PBS) and PTS dye (dissolved in PTS buffer) were mixed, and the Tm value was detected by Applied Biosystems ViiA7 real-time PCR instrument. The detection results are as shown in Table 2.

TABLE 2
Tm values for fibronectin type III domain fusion proteins

| Construct Names | Tm (° C.) |
|---|---|
| ScFv2-FN7-ScFv1 | 61, 73 |
| ScFv2-FN10-ScFv1 | 61, 71 |
| ScFv3-FN7-ScFv1 | 68 |
| ScFv3-FN10-ScFv1 | 66 |
| ScFv3-FN7-Fc | 66 |
| ScFv3-FN7-Fc-ScFv1 | 66, 72 |
| Fab2H-FN7-Scfv1 | 74, 83 |
| Fab2L-FN7-Scfv1 | 74, 83 |
| Fab1L-FN7-Scfv2 | 83 |
| Fab1L-FN10-Scfv2 | 73 |
| Fab1L-FN7-Scfv3 | 64, 81 |
| Fab1H-FN10-Scfv3 | 63, 77 |
| FN10-3a-GCSF | 59 |
| FN10-3g-GCSF | 61 |

The above are only the preferred embodiments of the present application and do not limit the present application in any form or substance. It should be pointed out that for those of ordinary skill in the art, without departing from the method of the present application, several improvements and supplements can be made, and these improvements and supplements should also be regarded as the protection scope of the present application. Anyone who is familiar with the profession, without departing from the spirit and scope of the present application, can make use of the technical content disclosed above to make minor changes, modifications, and evolutionary equivalent changes, which are all equivalent embodiments; at the same time, any changes, modifications and evolutions made to the above-mentioned embodiments based on the essential technology of the present application are still within the scope of the technical solutions of the present application.

What is claimed is:

1. A fibronectin type III domain fusion protein, comprising:
a fibronectin type III domain;
one or more linkers; and
a first physiologically active peptide inserted within a flexible loop formed between two adjacent β chains selected from the group consisting of AB loop, BC loop, CD loop, DE loop, EF loop or FG loop, of the fibronectin type III domain.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. The fibronectin type III domain fusion protein of claim 1, wherein at least one linker in the fusion protein is a flexible peptide, wherein the flexible peptide is a polypeptide having a flexible structure;
preferably, wherein each linker in the fusion protein is a flexible peptide;
preferably, wherein the flexible peptide consists of small molecular weight polar amino acids such as glycine (Gly), serine (ser), threonine (Thr), alanine (Ala), glutamic acid (Glu) or phenylalanine (Phe);
preferably, wherein the flexible peptide is selected from the group consisting of: (G4S)n, wherein n=1, 2, 3, 4 or 5; (Gly)₈, (Gly)₆, GGGSGGGGS, GGGGSGGGS, GSAGSAAGSGEF, KESGSVSSEQLAQFRSLD or EGKSSGSGSESKST.

18. (canceled)

19. (canceled)

20. (canceled)

21. The fibronectin type III domain fusion protein of claim 1, wherein at least one linker in the fusion protein is a rigid peptide, preferably the rigid peptide consists of α-helices;
preferably, wherein each linker in the fusion protein is a rigid peptide consisting of α-helices;
preferably, wherein an amino acid sequence of the rigid peptide consisting of α-helices is selected from the group consisting of: (EAAAK)ₙ, wherein n=1, 2, 3, 4, or 5; and A (EAAAK)ₙA (n=2-5).

22. (canceled)

23. (canceled)

24. (canceled)

25. (canceled)

26. (canceled)

27. (canceled)

28. (canceled)

29. (canceled)

30. (canceled)

31. (canceled)

32. The fibronectin type III domain fusion protein of claim 1, comprising an amino acid sequence shown in any one of SEQ ID NOs: 10, 12, 14, 16, 18, 20, 22, 24, 26, 28, 30, 32, 34, 36, 38, 40, 42, 44, 46, 48, 50, 52, 54, 56, 58, 60, 62, 74, 76, 78, 80, 82, 84, 86, 88, 90, 92, 94, 96, 98, 100, 102, 104, 106, 108, 110, 112, and 114.

33. A polynucleotide encoding the fibronectin type III domain fusion protein of claim 1, preferably comprising a nucleotide sequence selected from the group consisting of: SEQ ID NOs: 9, 11, 13, 15, 17, 19, 21, 23, 25, 27, 29, 31, 33, 35, 37, 39, 41, 43, 45, 47, 49, 51, 53, 55, 57, 59, 61, 73, 75, 77, 79, 81, 83, 85, 87, 89, 91, 93, 95, 97, 99, 103, 105, 107, 109, 111, and 113.

34. An expression vector comprising the polynucleotide of claim 33.

35. A host cell comprising the expression vector of claim 34.

36. The host cell of claim 35, wherein the host cell is a mammalian host cell transiently transfected with the expression vector of claim 34.

37. A method for preparing the fibronectin type III domain fusion protein of claim 1, comprising: culturing the mammalian host cell of claim 35 under conditions that permit expression of the fibronectin type III domain fusion protein; and collecting the fibronectin type III domain fusion proteins secreted from culture supernatants.

38. A pharmaceutical composition comprising the fibronectin type III domain fusion protein of claim 1 and a pharmaceutically acceptable carrier.

39. (canceled)

40. The pharmaceutical composition of claim 38, wherein the fibronectin type III domain is:
(a) a fibronectin type 7 III domain (FN7), wherein the FN is human FN7, in particular a FN7 having the sequence shown in SEQ ID NO: 2; alternatively, the FN is mouse FN7, in particular a FN7 having the sequence shown in SEQ ID NO: 70; or,
(b) a fibronectin type 10 III domain (FN10), wherein the FN10 is human FN10, in particular a FN10 having the sequence shown in SEQ ID NO: 4; alternatively, the FN10 is mouse FN10, in particular a FN10 having the sequence shown in SEQ ID NO: 72.

41. (canceled)

42. The pharmaceutical composition of claim 38, wherein the physiologically active peptide is selected from the group consisting of: a hormone, a cytokine, a vaccine antigen, an antigen protein, an enzyme, a growth factor, a transcription regulatory factor, a coagulation factor, a structural protein, a ligand protein and a receptor, an antibody or an antigen-binding fragment thereof, a toxic protein; human growth factor, human granulocyte colony stimulating factor, RSV F protein, OVA protein, a Fc, a Fab heavy chain, a Fab light chain or a scFv.

43. The fibronectin type III domain fusion protein of claim 17, further comprising a second physiologically active peptide, wherein the second physiologically active peptide is inserted within a flexible loop formed between two adjacent β chains of the fibronectin type III domain, preferably, the flexible loop is selected from the group consisting of AB loop, BC loop, CD loop, DE loop, EF loop or FG loop, of the fibronectin type III domain by the linker, and the second physiologically active peptide and the first physiologically active peptide are inserted at different positions of the fibronectin type III domain; or
wherein the second physiologically active peptide is linked to the N-terminal or the C-terminal of the fibronectin type III domain by the linker.

44. The fibronectin type III domain fusion protein of claim 21, further comprising a second physiologically active peptide, wherein the second physiologically active peptide is inserted within a flexible loop formed between two adjacent β chains of the fibronectin type III domain, preferably, the flexible loop is selected from the group consisting of AB loop, BC loop, CD loop, DE loop, EF loop or FG loop, of the fibronectin type III domain by the linker, and the second physiologically active peptide and the first physiologically active peptide are inserted at different positions of the fibronectin type III domain; or
wherein the second physiologically active peptide is linked to the N-terminal or the C-terminal of the fibronectin type III domain by the linker.

45. The fibronectin type III domain fusion protein of claim 1, wherein the fibronectin type III domain is a fibronectin 7^{th} type III domain (FN7) or a fibronectin 10^{th} type III domain (FN10);
preferably, wherein the FN7 is selected from human FN7 or mouse FN7; more preferably, the human FN7 comprising an amino acid sequence having at least 90% identity with the amino acid sequence shown in SEQ ID NO: 2; more preferably, the mouse FN7 comprising an amino acid sequence having at least 90% identity with the amino acid sequence as shown in SEQ ID NO: 70;
preferably, wherein the FN10 is selected from human FN10 or mouse FN10; more preferably, the human FN10 comprising an amino acid sequence having at least 90% identity with the amino acid sequence shown in SEQ ID NO: 4; more preferably, the mouse FN10 comprising an amino acid sequence having at least 90% identity with the amino acid sequence shown in SEQ ID NO: 72.

46. The fibronectin type III domain fusion protein of claim 17, wherein the fibronectin type III domain is a fibronectin 7^{th} type III domain (FN7) or a fibronectin 10^{th} type III domain (FN10);
preferably, wherein the FN7 is selected from human FN7 or mouse FN7; more preferably, the human FN7 comprising an amino acid sequence having at least 90% identity with the amino acid sequence shown in SEQ ID NO: 2; more preferably, the mouse FN7 comprising an amino acid sequence having at least 90% identity with the amino acid sequence as shown in SEQ ID NO: 70;
preferably, wherein the FN10 is selected from human FN10 or mouse FN10; more preferably, the human FN10 comprising an amino acid sequence having at least 90% identity with the amino acid sequence shown in SEQ ID NO: 4; more preferably, the mouse FN10 comprising an amino acid sequence having at least 90% identity with the amino acid sequence shown in SEQ ID NO: 72.

47. The fibronectin type III domain fusion protein of claim 21, wherein the fibronectin type III domain is a fibronectin 7^{th} type III domain (FN7) or a fibronectin 10^{th} type III domain (FN10);
preferably, wherein the FN7 is selected from human FN7 or mouse FN7; more preferably, the human FN7 comprising an amino acid sequence having at least 90% identity with the amino acid sequence shown in SEQ ID NO: 2; more preferably, the mouse FN7 comprising an amino acid sequence having at least 90% identity with the amino acid sequence as shown in SEQ ID NO: 70;
preferably, wherein the FN10 is selected from human FN10 or mouse FN10; more preferably, the human FN10 comprising an amino acid sequence having at least 90% identity with the amino acid sequence shown in SEQ ID NO: 4; more preferably, the mouse FN10 comprising an amino acid sequence having at least 90% identity with the amino acid sequence shown in SEQ ID NO: 72.

48. The fibronectin type III domain fusion protein of claim 17, wherein the first physiologically active peptide is selected from the group consisting of a hormone, a cytokine, a vaccine antigen, an antigen protein, an antigen protein acceptor, an interleukin-fusion protein, a growth factor, a transcription regulatory factor, a coagulation factor, a structural protein, a ligand protein, a ligand protein receptor, a receptor antagonist, a cell surface antigen, an antigen-binding fragment, a toxic protein, human growth factor, a colony stimulating factor, a viral-derived antigenic protein, a Fc, growth hormone releasing peptide, an interferon, an interferon receptor, a monoclonal antibody, a polyclonal antibody and an antibody fragment, glucagon-like peptide, a G protein-coupled receptor, an interleukin, an interleukin receptor, an enzyme, an interleukin binding protein, a cytokine binding protein, a macrophage activating factor, a B cell factor, a T cell factor, protein A, an allergy inhibitor, a cell necrosis glycoprotein, an immunotoxin, a lymphotoxin, a tumor necrosis factor, a tumor suppressor, a metastasis growth factor, α-1 antitrypsin, albumin, α-lactalbumin, apolipoprotein-E, erythropoietin, a highly glycosylated erythropoietin, an angiopoietin, hemoglobin, thrombin, a thrombin receptor activating peptide, thrombomodulin, factor VII, factor VIIa, factor VIII, factor IX, factor XIII, a plasminogen activator, a fibrin-binding peptide, urokinase, streptokinase, hirudin, protein C, C-reactive protein, a renin inhibitor, a collagenase inhibitor, a superoxide dismutase, leptin, a platelet-derived growth factor, an epithelial growth factor, an epidermal growth factor, an angiostatin, an angiotensin, a bone growth factor, a bone stimulating protein, calcitonin, insulin, atrial peptide hormone, cartilage-inducing factor, elcatonin, a connective tissue activating factor, a tissue factor pathway inhibitor, follicle-stimulating hormone, luteinizing hormone, luteinizing hormone releasing hormone, a nerve growth factor, parathyroid hormone, relaxin, secretin, a stomatomedin, an insulin-like growth factor, an adreno cortical hormone, glucagon, cholecystokinin, pancreatic polypeptide, gastrin releasing peptide, a corticotropin releasing factor, thyroid stimulating hormone, an autocrine motility factor, lactoferrin, and tubocurarine;
preferably, wherein the first physiologically active peptide is selected from the group consisting of: human growth factor, human granulocyte colony stimulating factor, RSV F protein, OVA, a Fc, a Fab heavy chain, a Fab light chain and a scFv.

49. The fibronectin type III domain fusion protein of claim 21, wherein the first physiologically active peptide is selected from the group consisting of a hormone, a cytokine, a vaccine antigen, an antigen protein, an antigen protein acceptor, an interleukin-fusion protein, a growth factor, a transcription regulatory factor, a coagulation factor, a structural protein, a ligand protein, a ligand protein receptor, a receptor antagonist, a cell surface antigen, an antigen-binding fragment, a toxic protein, human growth factor, a colony stimulating factor, a viral-derived antigenic protein, a Fc, growth hormone releasing peptide, an interferon, an interferon receptor, a monoclonal antibody, a polyclonal antibody and an antibody fragment, glucagon-like peptide, a G protein-coupled receptor, an interleukin, an interleukin receptor, an enzyme, an interleukin binding protein, a cytokine binding protein, a macrophage activating factor, a B cell factor, a T cell factor, protein A, an allergy inhibitor, a cell necrosis glycoprotein, an immunotoxin, a lymphotoxin, a tumor necrosis factor, a tumor suppressor, a metastasis growth factor, α-1 antitrypsin, albumin, α-lactalbumin, apolipoprotein-E, erythropoietin, a highly glycosylated erythropoietin, an angiopoietin, hemoglobin, thrombin, a thrombin receptor activating peptide, thrombomodulin, factor VII, factor VIIa, factor VIII, factor IX, factor XIII, a plasminogen activator, a fibrin-binding peptide, urokinase, streptokinase, hirudin, protein C, C-reactive protein, a renin inhibitor, a collagenase inhibitor, a superoxide dismutase, leptin, a platelet-derived growth factor, an epithelial growth factor, an epidermal growth factor, an angiostatin, an angiotensin, a bone growth factor, a bone stimulating protein, calcitonin, insulin, atrial peptide hormone, cartilage-inducing factor, elcatonin, a connective tissue activating factor, a tissue factor pathway inhibitor, follicle-stimulating hormone, luteinizing hormone, luteinizing hormone releasing hormone, a nerve growth factor, parathyroid hormone, relaxin, secretin, a stomatomedin, an insulin-like growth factor, an adreno cortical hormone, glucagon, cholecystokinin, pancreatic polypeptide, gastrin releasing peptide, a corticotropin releasing factor, thyroid stimulating hormone, an autocrine motility factor, lactoferrin, and tubocurarine;
preferably, wherein the first physiologically active peptide is selected from the group consisting of: human growth factor, human granulocyte colony stimulating factor, RSV F protein, OVA, a Fc, a Fab heavy chain, a Fab light chain and a scFv.
